(19) United States
(12) Patent Application Publication
(10) Pub. No.: US 2025/0369958 A1
(43) Pub. Date: Dec. 4, 2025

(54) METHODS FOR PREDICTION AND TREATMENT OF LIMB-GIRDLE MUSCULAR DYSTROPHY

(71) Applicants: Gabriel Haller, St. Louis, MO (US); Conrad Weihl, St. Louis, MO (US); Chengcheng Li, St. Louis, MO (US)

(72) Inventors: Gabriel Haller, St. Louis, MO (US); Conrad Weihl, St. Louis, MO (US); Chengcheng Li, St. Louis, MO (US)

(73) Assignee: Washington University, St. Louis, MO (US)

(21) Appl. No.: 19/222,473

(22) Filed: May 29, 2025

Related U.S. Application Data
(60) Provisional application No. 63/653,397, filed on May 30, 2024.

(51) Int. Cl. G01N 33/50; C12N 15/10

(52) U.S. Cl. CPC G01N 33/5091; C12N 15/1058

(57) ABSTRACT

Methods of generating a limb-girdle muscular dystrophy (LGMD) functional score, as well as methods of predicting and treating LGMD are provided. The present disclosure teaches methods of generating a LGMD functional score through single amino acid mutagenesis and deep mutation scanning (DMS) data including a mutant reads high expression and a mutant reads low expression.

### CROSS-REFERENCE TO RELATED APPLICATIONS

This application claims the benefit of priority to U.S. Provisional Application Ser. No. 63/653,397 filed on May 30, 2024, which is incorporated herein by reference in its entirety.

### STATEMENT REGARDING FEDERALLY SPONSORED RESEARCH OR DEVELOPMENT

This invention was made with government support under AR078942 awarded by the National Institutes of Health. The government has certain rights in the invention.

### MATERIAL INCORPORATED-BY-REFERENCE

Not applicable.

### FIELD OF THE INVENTION

The present disclosure generally relates to muscular dystrophy disease prediction and treatment.

### BACKGROUND OF THE INVENTION

Recessive mutations in β-sarcoglycan (SGCB) cause limb-girdle muscular dystrophy type R4/2E (LGMDR4/2E), resulting in muscle wasting, progressive weakness, degeneration of skeletal muscle, and often premature death. β-Sarcoglycan is a key component of the dystrophin-associated protein complex. In muscle cells, the dystrophin-associated protein complex localizes to the membrane and connects the intracellular cytoskeleton to the extracellular matrix, allowing for coordinated force production in muscle. The dystrophin complex also acts as a membrane stabilizer during muscle contraction to prevent contraction-induced damage. The sarcoglycan subcomplex is composed of 4 single-pass transmembrane proteins: α-sarcoglycan, β-sarcoglycan, γ-sarcoglycan, and δ-sarcoglycan. The sarcoglycan subunits assemble and translocate within the myofiber as a complex, and loss of any individual subunit due to loss-of-function mutations adversely affects the stability and trafficking of the unmutated sarcoglycan proteins, leading to what is referred to as sarcoglycanopathy. A handful of missense pathogenic variants have been identified in each sarcoglycan. These missense mutations lead to a failure in sarcolemmal localization and sarcoglycan complex formation. Currently, no crystal or cryo-EM structure exists for this essential membrane complex that is critical to human health. Thus, whether missense mutations in sarcoglycans destabilize the protein, alter its trafficking to the sarcolemma, or affect its interactions with its sarcoglycan partners is not fully understood.

Clinical diagnosis of sarcoglycan-deficient LGMD currently requires histopathologic assessment of a patient's muscle biopsy for cell surface-localized sarcoglycan complex proteins or biochemical assessment of the protein's presence. Loss of one sarcoglycan subunit often secondarily leads to disruption of the entire sarcoglycan complex, further confounding a true diagnosis without genetic confirmation. Because of this apparent overlap in phenotype between the sarcoglycanopathies, and the phenotypic heterogeneity of other genetically defined LGMDs, obtaining a genetic diagnosis can be challenging. Genotype-phenotype correlations are emerging within the sarcoglycanopathies. For example, some mutations in SGCB are associated with late disease onset in the second decade of life, whereas other mutations result in early adolescent onset. Moreover, the challenge of diagnosing patients with LGMDR4/2E before symptom onset or early in the course of the disease has the potential to enable the use of preventative gene therapy or other therapeutics, making the disorder highly clinically actionable.

Missense changes constitute the majority of variants observed in patients with LGMD, and, in most instances, particularly for recessive conditions, there is insufficient evidence to classify variants as pathogenic or benign, resulting in the designation as a variant of unknown significance (VUS). VUSs present a diagnostic dilemma to patients and clinicians. At present there is no systemized path forward for “variant resolution.” The American College of Medical Genetics and Genomics (ACMG) has proposed strict criteria to assert the pathogenicity of a disease variant. One underutilized criterion in LGMD genes is PS3 (strong evidence, i.e. high accuracy in defining pathogenicity). The use of PS3 requires that a variant be assessed using a well-established in vitro or in vivo functional study to support a damaging effect on the gene or gene product. This process requires the development of gene-specific functional assays, making an individualized single patient variant resolution pipeline labor intensive and prohibitively expensive.

### SUMMARY OF THE INVENTION

Among the various aspects of the present disclosure is the provision of methods for predicting the development of limb-girdle muscular dystrophy.

In one aspect of the present disclosure, a method of generating a limb-girdle muscular dystrophy (LGMD) functional score is provided. The method comprises: providing a cell sample from the subject; performing a single amino acid mutagenesis on a target protein in the cell sample; performing a deep mutation scan (DMS) on the target protein following the single amino acid mutagenesis; determining a mutant reads high expression, defined as a number of mutant read counts in cells of the cell sample having a high cell surface level of the target protein; determining a mutant reads low expression, defined as a number of mutant read counts in cells of the cell sample having a low cell surface level of the target protein; and generating a LGMD functional score for the target protein based on the mutant reads high expression and the mutant reads low expression.

In some embodiments, the target protein is a SGC protein. In some embodiments, the SGC protein is a SGCB protein. In some embodiments, the single amino acid mutagenesis is derived from an amino acid change requiring at least one nucleotide change. In some embodiments, the LGMD functional score is generated by:

$$Functional score = {log}_{10} \frac{mutant reads high expression}{mutant reads low expression} .$$

In some embodiments, the LGMD functional score ranges from a score of −3 to a score of 1.5, a negative LGMD functional score indicates a deleterious variant and a positive LGMD functional score indicates a neutral variant, and/or the LGMD functional score of less than −2 indicates severe LGMD and the LGMD functional score of greater than −2 indicates mild LGMD.

In another aspect of the present disclosure, a method of treating limb-girdle muscular dystrophy (LGMD) in a subject in need thereof is provided. The method comprises: providing a cell sample from the subject; performing a single amino acid mutagenesis on a target protein in the cell sample; performing a deep mutation scan (DMS) on the target protein following the single amino acid mutagenesis; determining a mutant reads high expression, defined as a number of mutant read counts in cells of the cell sample having a high cell surface level of the target protein; determining a mutant reads low expression, defined as a number of mutant read counts in cells of the cell sample having a low cell surface level of the target protein; generating a LGMD functional score for the target protein based on the mutant reads high expression and the mutant reads low expression; and determining a gene therapy treatment for the subject based on the LGMD functional score.

In some embodiments, the target protein is a SGC protein. In some embodiments, the SGC protein is a SGCB protein. In some embodiments, the single amino acid mutagenesis is derived from an amino acid change requiring at least one nucleotide change. In some embodiments, the LGMD functional score is generated by:

$$Functional score = {log}_{10} \frac{mutant reads high expression}{mutant reads low expression} .$$

In some embodiments, the LGMD functional score ranges from a score of −3 to a score of 1.5, a negative LGMD functional score indicates a deleterious variant and a positive LGMD functional score indicates a neutral variant, and/or the LGMD functional score of less than −2 indicates severe LGMD and the LGMD functional score of greater than −2 indicates mild LGMD.

In a further aspect of the present disclosure, a method for determining limb-girdle muscular dystrophy (LGMD) severity in a subject in need thereof is provided. The method comprises: providing a cell sample from the subject; performing a single amino acid mutagenesis on a target protein in the cell sample; performing a deep mutation scan (DMS) on the target protein following the single amino acid mutagenesis; determining a mutant reads high expression, defined as a number of mutant read counts in cells of the cell sample having a high cell surface level of the target protein; determining a mutant reads low expression, defined as a number of mutant read counts in cells of the cell sample having a low cell surface level of the target protein; generating a LGMD functional score for the target protein based on the mutant reads high expression and the mutant reads low expression; and determining a LGMD severity for the subject based on the LGMD functional score.

In some embodiments, the LGMD severity comprises at least one of an age at loss of ambulation LGMD severity and an age at onset LGMD severity. In some embodiments, the LGMD functional score is generated by:

$$Functional score = {log}_{10} \frac{mutant reads high expression}{mutant reads low expression} .$$

In some embodiments, the LGMD functional score ranges from a score of −3 to a score of 1.5; a negative LGMD functional score indicates a deleterious variant and a positive LGMD functional score indicates a neutral variant; and the LGMD functional score of less than −2 indicates severe LGMD and the LGMD functional score of greater than −2 indicates mild LGMD.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings described herein are for illustrative purposes only. The drawings are not intended to limit the scope of the present teachings in any way.

FIG. 1 is a schematic showing the process to accurately predict pathogenicity in limb-girdle muscular dystrophy. A mutation library is created and introduced into cells. SGC cell surface expression is assessed and cells are FAC sorted by immunofluorescence. The function sore is calculated and used to predict pathogenicity.

FIG. 2A is a schematic showing the first step for generating and testing β-sarcoglycan (SGCB) variant function. Mutation libraries are generation by cloning synthesized oligos into a wildtype backbone.

FIG. 2B is a schematic showing the creation and transduction of lentiviral libraries derived from SGCD mutant plasmid libraries from FIG. 2A.

FIG. 2C is a set of immunofluorescent images of cells transduced with lentiviral SGCD mutants (YFP). Cells with pathogenic variants (G167S; bottom) fail to effectively transport intracellular SGCB (green) to the cell surface (red), while WT (top) demonstrates robust total protein and cell surface expression of SGCB.

FIG. 2D is a FACS plot showing WT (red) and G167S (purple) SGCB transduced cells sorted for HA into four bins.

FIG. 2E is a schematic showing the sequencing of the WT and G167S transduced cells which were sorted into four bins in FIG. 2D.

FIG. 2F is a table showing the calculation of functional scores from mutation prevalence in each bin of HA staining. The resulting functional score is negative for deleterious variants or positive for functionally neutral variants.

FIG. 3A is a schematic showing a Helices (green) and β sheets (orange) as predicted from the AlphaFold2 multimer model of SGCB modeled with SGCA, SGCG, and SGCD. A heat map shows the functional score of biological replicate median values for each amino acid change. Scores range from damaging (red) to benign variants (blue). Missing or low confidence data are shown in yellow. Synonymous changes are bounded in black boxes. Average HA functional score (constraint score) per position (318 amino acids) is shown as a heatmap with each row being a different amino acid substitution labeled with the amino acid abbreviation.

FIG. 3B is a histogram showing HA functional scores demonstrate a bimodal distribution with synonymous variants (blue) showing a narrow range of scores around 1 (i.e., enriched in HA bin 4).

FIG. 3C is a scatter plot showing the correlation among biological replicates of HA-stained ADG-HEK cells transduced with SGCB libraries (libraries A-F are included) and ClinVar pathogenic variants (red), benign variants (green), and synonymous variants (yellow) highlighted.

FIG. 4A is a graph showing the concordance of functional scores (y-axis) with existing clinical classifications. The functional scores for patient missense variants from ClinVar databases are shown by variant classification (x axis).

FIG. 4B is a graph of receiver operator curves (ROC) predicting pathogenic or benign ClinVar classification (33 variants) for the functional score generated (DMS, REVEL, CADD, or PolyPhen).

FIG. 4C is a scatter plot showing the concordance of computational predictor REVEL with DMS scores. Quadrants are distinguished by color scores of greater than or less than 0.75 and DMS scores of greater than or less than 0 and based on clinical classification as pathogenic/likely pathogenic (red) or benign/likely benign (green).

FIG. 5A is a set of flow cytometry plots showing the relationship between HA-immunofluorescence (HA-Alexa647) and YFP expression level (FITC, y axis) for ADG-HEK cells transduced with lentivirus to express either WT or mutant SGCB.

FIG. 5B is a bar graph quantifying the number of YFP-positive cells that also demonstrated positive HA cell surface staining.

FIG. 5C is a bar graph showing the average age at onset (AAO; black) and age at loss of ambulation (ALA; grey) for individuals homozygous for given variants in SGCB.

FIG. 5D is a plot of Cox's proportional hazard curves for loss of ambulation among genetically diagnosed patients with LGMD with SGCB pathogenic variants with HA functional scores that sum less than −2 (severe) or more than −2 (milder).

FIG. 6 is a schematic showing structural insights from SGCB deep mutational scanning data. AlphaFold2 multimer model of SGCB-SGCA-SGCD-SGCG protein complex with SGCB surface colored, with average functional score of amino acid changes at each position and SGCA, SGCD, and SGCG colored in light gray. Ball-and-stick model of SGCB structure (modeled with SGCA, SGCD, and SGCG) highlighting the increased deleteriousness of amino acid changes at positions with side chains with multiple intermolecular interactions.

FIG. 7A is a plot showing the distribution of HA functional scores for amino acid changes possible with a single nucleotide change versus those possible only with multi-nucleotide changes.

FIG. 7B is a density plot showing the minimum number of nucleotide changes required for each amino acid to be changed either to a nonfunctional (score, <˜0.5) amino acid (red) or a different functional (score, >−0.5) amino acid (blue). T tests were used to test for differences in distribution between groups.

FIG. 8A is a set of immunofluorescence images of HEK cells transduced with SGC genes (from top to bottom: SGCB, SGCB+A, SGCB+D, SGCB+G) via lentivirus and measured for total expression (green, left), cell-surface expression (red, middle), and merge (yellow, right).

FIG. 8B is a set of immunofluorescence images of HEK cells transduced with SGC genes (from top to bottom: SGCB+A+D, SGCB+A+G, SGCB+D+G, SGCB+A+D+G) via lentivirus and measured for total expression (green, left), cell-surface expression (red, middle), and merge (yellow, right).

FIG. 8C is a graph quantifying the percent expression of SGC extracellular expression (Alexa647, red) from FIG. 8A and FIG. 8B.

FIG. 9A is a set of western blot images of stable ADG-HEK cells. HEK293 cells were transduced with three lentiviruses expressing SGCA, SGCD and SGCG, respectively. Cells were single-cell sorted using FACS and grown to generate clonal cells lines. Cell lines positive for lentiviral DNA from each gene (1, 4, 10, 14) were screened for protein expression by western blot at three different passages after transduction (P1, P2 and P3). ADG-HEK line #10 was used for all DMS experiments.

FIG. 9B is a set of western blot images of ADG-HEK cells (line #10, passage #5) which were transduced with SGCB wild-type lentivirus and assessed for SGCA, SGCB, SGCD, and SGCG protein level.

FIG. 10 is a flow cytometry plot of ADG=HEK cells transduced with SGCB-WT and co-stained for cell surface SGCB and SGCA. YFP positive cells were first gated to exclude non-transduced cells, and then quadrant gates were drawn based on negative control cells (i.e. SGCB-WT transduced cells without antibody staining, or stained with HA-Pacific blue or SGCA-Alexa647), 77.7% of cells were positive for both SGCB and SGCA cell surface protein. There was a strong positive correlation between the amount of SGCB cell surface protein and SGCA cell surface protein. A larger proportion of cells were SGCB positive (91.9%) than were SGCA positive (80.63%). This may simply be due to differences in background fluorescence for the two antibodies and the requisite cutoffs for positivity as a result.

FIG. 11A is a set of flow cytometry plots showing the relationship between HA-immunofluorescence (HAAlexa647) and YFP expression level (FITC, y-axis) for ADG-HEK cells transduced with lentivirus to express either wildtype (WT) or mutant SGCB.

FIG. 11B is a graph showing the quantification of the number of YFP-positive cells that also demonstrated positive HA cell-surface staining from FIG. 11A. *** p<0.001 compared to WT.

FIG. 12A is a set of immunofluorescent images of cell surface expression of SGCB for single SGCB variants. ADG-HEK cells were transduced with single SGCB variants via lentivirus and then measured for total SGCB (green, left) expression and cell-surface SGCB (red, middle) expression.

FIG. 12B is a set of immunofluorescent images of cell surface expression of SGCA for single SGCB variants. ADG-HEK cells were transduced with single SGCB variants via lentivirus and then measured for total SGCB (green, left) expression and cell-surface SGCA (red, middle) expression.

FIG. 13A is a set of flow cytometry plots showing the relationship between SGCA-immunofluorescence and YFP expression level (FITC, y-axis) for ADG-HEK cells transduced with lentivirus to express either wildtype (WT) or mutant SGCB.

FIG. 13B is a graph quantifying Quantification of the number of YFP-positive cells that also demonstrated positive SGCA cell-surface staining from FIG. 13A.

FIG. 13C is a graph showing the correlation between the percentage of positive cells for single variant transduced cells for cell-surface SGCB protein and cell-surface SGCA protein.

FIG. 14A is a schematic showing a functional effect map of SGCA. The top bars indicate alpha helices (green) and beta-sheets (orange) as predicted from the AlphaFold multimer model of SGCB modelled with SGCA, SGCG and SGCD. A heatmap which shows SGCA functional score of biological replicate median values. Scores range from damaging (red) to benign variants (blue). Missing or low confidence data are shown in yellow. Synonymous changes are bounded in black boxes. Each row represents a different amino acid substitution labeled with the amino acid abbreviation. The bottom row represents the average SGCA functional score per position (blue, neutral; red, deleterious).

FIG. 14B is a histogram of SGCA functional scores demonstrate a bimodal distribution with synonymous variants (blue) showing a narrow range of scores around 1 (i.e. enriched in SGCA bin 4).

FIG. 14C is a plot showing the correlation between biological replicates of SGCA-stained ADG-HEK cells transduced with SGCB libraries (libraries A-F are included) with ClinVar pathogenic variants (red) and benign variants (orange) highlighted.

FIG. 15A is a plot showing the predicted functional scores (y axes) for patient missense variants from ClinVar/Leiden databases by variant classification (x axis).

FIG. 15B is a plot of receiver operator curves (ROC) predicting Pathogenic or Benign classification (27 variants) for the functional score predicted by alignment to SGCB (DMS), REVEL, CADD or PolyPhen.

FIG. 16 is a diagram of SGCB sub-libraries. Libraries of oligos, each containing a single codon encoded as ‘NNN’ were purchased from IDT and named pools A-F and covered the entirety of the SGCB coding sequence with 42 bp of overlapping sequence between each pair of sub-library.

### DETAILED DESCRIPTION OF THE INVENTION

The present disclosure is based, at least in part, on disease prediction and treatment determination of limb-girdle muscular dystrophy based on alteration in the SGCB protein.

As described herein, disease predictions have been generated for all possible protein-altering single nucleotide variants in the gene SGCB, which causes recessive limb-girdle muscular dystrophy type 4R/2E. By performing single amino acid mutagenesis and deep mutation scanning, a resulting functional score enables accurate prediction of pathogenicity for limb-girdle muscular dystrophy and informs a treatment determination, particularly with respect to what patients should be given certain gene-specific therapy based on the disease progression prediction.

The present disclosure describes the effect of all possible missense SGCB variants using single amino acid saturation mutagenesis to generate libraries comprising every possible missense, synonymous, and nonsense variant. Functional scores for each variant were calculated for YFP-SGCB-HA cell surface expression and SGCA cell surface expression as the logio ratio of the variant's frequency of high expression divided by its frequency of low expression, such that deleterious variants should score negatively and neutral variants positively.

Disclosed is the method of high-throughput functional assays which can accurately measure the effect of protein-coding genetic variation in the LGMD gene SGCB. The map of functional effects presented has the ability to improve classification of variants observed in patients with LGMD and aid the understanding of the structure of an important member of the dystrophin-associated protein complex. When used together with available lines of evidence, these results add confidence to variant interpretation and potentially allow patients with pathogenic variants to be treated with gene-specific therapeutics for which they would have otherwise been ineligible.

The sarcoglycan genes and SGCB in particular are among the most frequently mutated genes underlying LGMD. Like many recessive disease genes, affected patients carrying biallelic loss-of-function variants (i.e., premature termination codons or indels resulting in frame shifts) provide strong evidence for variant pathogenicity. Disclosed is the integration of massively parallel assays of SGCB function, SGCB cell surface expression and SGCA cell surface expression, and generated a near-complete map of the functional effect of missense variants in the LGMD gene SGCB.

Measured functional scores for variants are highly accurate in predicting the pathogenicity of known disease-causing variants, outperforming the newest prediction algorithms. The functional measurements were highly consistent with expert-reviewed variant classification records from the ClinVar database and the Leiden genetic variant database that often use sarcolemmal expression in patient muscle tissue as their functional evidence. This disclosure provides functionally relevant evidence to be used in variant resolution, which satisfies the requirements set forth by the ACMG to add strong evidence for the pathogenicity of variants with negative functional scores in assays (PS3 criteria). The functional effect maps presented may allow for the potential reclassification of variants with limited evidence present in clinical databases. The disclosed measured functional scores were a better predictor of known pathogenic and benign variants than Polyphen2, CADD, or REVEL scores.

Additionally, disease severity, as measured by age at loss of ambulation, is related to functional score in the assay suggesting that cell surface expression of SGCB and the SGC protein complex may be a quantitative trait that determines, in part, the stability of the sarcolemma and the integrity of muscle cells over the course of a lifetime.

The pattern of deleterious amino acid changes across the SGCB gene closely mirrored the predicted protein structure produced using AlphaFold2. The co-occurrence of deleterious amino acid changes within predicted β sheets highlighted the importance of interprotein interactions, particularly between SGCB and SGCD, in producing a functional protein complex capable of being assembled and transported to the cell membrane. Intriguingly, there was minimal effect when amino acids within the intracellular domain or transmembrane domain of SGCB were changed. Overall, the scores and their pattern corroborate the predicted AlphaFold2 structure and improve understanding of the domains and interactions important for SGCB function. This was further demonstrated by the ability to predict the pathogenicity of SGCD and SGCG variants with high accuracy using SGCB functional scores and knowledge of the structural relationship between the 3 proteins. By aligning the 3 genes' protein structures and superimposing SGCB scores on SGCD and SGCG, further insight was gained into the importance of interprotein contacts by accurately predicting pathogenic variants in these two related proteins.

Overall, SGCB is moderately tolerant of protein-altering genetic variation, with 16% of single-nucleotide missense variants demonstrating nonfunctional scores (score, <−0.5). This number jumps to 30% when considering all possible amino acid changes, however, implying that evolutionary forces lead to the selection of specific amino acids and even specific codons with fewer nonfunctional alleles reachable by single nucleotide changes. The bimodal distribution of functional scores is similar to previous deep-mutational scanning reports, with most SGCB missense variants demonstrating either a clear nonfunctional score or a neutral score.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| | | Bases | Complementary |
|---|---|---|---|
| Base | Name | Represented | Base |
| A | Adenine | A | T |
| T | Thymidine | T | A |
| U | Uridine(RNA | U | A |
| | only) | | |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product—consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA: DNA sequence can be determined using the following formula: Tₘ=81.5° C.+16.6 (log 10 [Na⁺])+0.41 (fraction G/C content)−0.63 (% formamide)−(600/1). Furthermore, the Tₘ of a DNA: DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain | |
|---|---|
| Characteristic | Amino Acid |
| Aliphatic Non-polar | G A P I L V |
| Polar-uncharged | C S T M N Q |
| Polar-charged | D E K R |
| Aromatic | H F W Y |
| Other | N Q D E |

Conservative Substitutions I

| Side Chain | |
|---|---|
| Characteristic | Amino Acid |
| Non-polar | |
| (hydrophobic) | |
| A. Aliphatic: | A L I V P |
| B. Aromatic: | F W |
| C. Sulfur- | |
| containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively | D E |
| Charged (Acidic): | |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33 (5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, gene and/or protein expression signals can be modulated (e.g., reduced, eliminated, or enhanced) using genome editing.

As described herein, activity, signals, expression, or function can be modulated (e.g., reduced, eliminated, or enhanced) using genome editing (e.g., upregulate, downregulate, overexpress, underexpress, express (e.g., transgenic expression), knock in, knock out, knockdown).

Processes for genome editing are well known; see e.g., Aldi 2018 Nature Communications 9 (1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of gene/protein expression/signaling by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications to target cells by the removal or addition of signals (e.g., activate (e.g., CRISPRa), upregulate, overexpress, downregulate).

For example, the methods as described herein can comprise a method for altering a target polynucleotide sequence in a cell comprising contacting the polynucleotide sequence with a clustered regularly interspaced short palindromic repeats-associated (Cas) protein.

### Gene Therapy and Genome Editing

Gene therapies are rapidly advancing and in some embodiments include inserting a functional gene with a viral vector. Improvement to the landscape for gene therapies and gene therapy clinical trials is ongoing (see, e.g., the most recent quarterly data breakdown from Alliance for Regenerative Medicine).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

Gene therapy strategies.

| | Strategy |
|---|---|
| Viral Vectors | |
| Retroviruses | Retroviruses are RNA viruses |
| | transcribing their single-stranded |
| | genome into a double-stranded DNA |
| | copy, which can integrate into host |
| | chromosome |
| Adenoviruses | Ad can transfect a variety of quiescent |
| (Ad) | and proliferating cell types from |
| | various species and can mediate |
| | robust gene expression |
| Adeno- | Recombinant AAV vectors contain no |
| associated | viral DNA and can carry ~4.7 kb of |
| Viruses (AAV) | foreign transgenic material. They are |
| | replication defective and can replicate |
| | only while coinfecting with a helper virus |
| Non-viral vectors | |
| plasmid DNA | pDNA has many desired characteristics |
| (pDNA) | as a gene therapy vector; there are no |
| | limits on the size or genetic |
| | constitution of DNA, it is relatively |
| | inexpensive to supply, and unlike |
| | viruses, antibodies are not generated |
| | against DNA in normal individuals |
| RNAi | RNAi is a powerful tool for gene |
| | specific silencing that could be |
| | useful as an enzyme reduction therapy |
| | or means to promote read-through of a |
| | premature stop codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Comprehensive Functional Characterization of SGCB Coding Variants Predicts Pathogenicity in Limb-Girdle Muscular Dystrophy Type R4/2E

### Introduction

Recessive mutations in β-sarcoglycan (SGCB) cause limb-girdle muscular dystrophy type R4/2E (LGMDR4/2E), resulting in muscle wasting, progressive weakness, degeneration of skeletal muscle, and often premature death. β-Sarcoglycan is a key component of the dystrophin-associated protein complex. In muscle cells, the dystrophin-associated protein complex localizes to the membrane and connects the intracellular cytoskeleton to the extracellular matrix, allowing for coordinated force production in muscle. The dystrophin complex also acts as a membrane stabilizer during muscle contraction to prevent contraction-induced damage. The sarcoglycan subcomplex is composed of 4 single-pass transmembrane proteins: α-sarcoglycan, β-sarcoglycan, γ-sarcoglycan, and δ-sarcoglycan. The sarcoglycan subunits assemble and translocate within the myofiber as a complex, and loss of any individual subunit due to loss-of-function mutations adversely affects the stability and trafficking of the unmutated sarcoglycan proteins, leading to what is referred to as sarcoglycanopathy. A handful of missense pathogenic variants have been identified in each sarcoglycan. These missense mutations lead to a failure in sarcolemmal localization and sarcoglycan complex formation. Currently, no crystal or cryo-EM structure exists for this essential membrane complex that is critical to human health. Thus, whether missense mutations in sarcoglycans destabilize the protein, alter its trafficking to the sarcolemma, or affect its interactions with its sarcoglycan partners is not fully understood.

Clinical diagnosis of sarcoglycan-deficient LGMD currently requires histopathologic assessment of a patient's muscle biopsy for cell surface-localized sarcoglycan complex proteins or biochemical assessment of the protein's presence. Loss of one sarcoglycan subunit often secondarily leads to disruption of the entire sarcoglycan complex, further confounding a true diagnosis without genetic confirmation. Because of this apparent overlap in phenotype between the sarcoglycanopathies, and the phenotypic heterogeneity of other genetically defined LGMDs, obtaining a genetic diagnosis can be challenging. Genotype-phenotype correlations are emerging within sarcoglycanopathies. For example, some mutations in SGCB are associated with late disease onset in the second decade of life, whereas other mutations result in early adolescent onset. Moreover, the challenge of diagnosing patients with LGMDR4/2E before symptom onset or early in the course of the disease has the potential to enable the use of preventative gene therapy or other therapeutics, making the disorder highly clinically actionable.

Missense changes constitute the majority of variants observed in patients with LGMD, and, in most instances, particularly for recessive conditions, there is insufficient evidence to classify variants as pathogenic or benign, resulting in the designation as a variant of unknown significance (VUS). VUSs present a diagnostic dilemma to patients and clinicians. At present there is no systemized path forward for “variant resolution.” The American College of Medical Genetics and Genomics (ACMG) has proposed strict criteria to assert the pathogenicity of a disease variant. One underutilized criterion in LGMD genes is PS3 (strong evidence, i.e. high accuracy in defining pathogenicity). The use of PS3 requires that a variant be assessed using a well-established in vitro or in vivo functional study to support a damaging effect on the gene or gene product. This process requires the development of gene-specific functional assays, making individualized single patient variant resolution pipeline labor intensive and prohibitively expensive. To address this current gap in variant resolution and assess the function effect of LGMD missense variants, deep mutational scanning (DMS) was employed to measure the effects of all possible missense variants of the SGCB gene.

### Results

### In Vitro Assay of SGCB Variant Function.

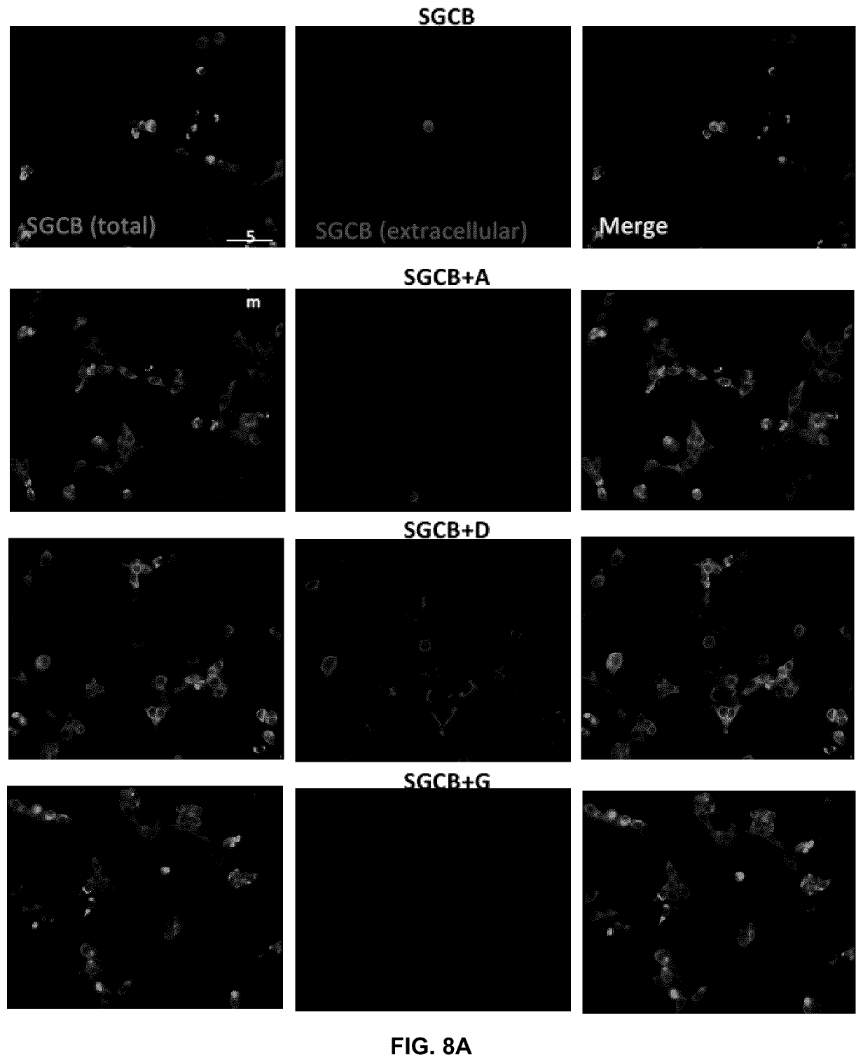

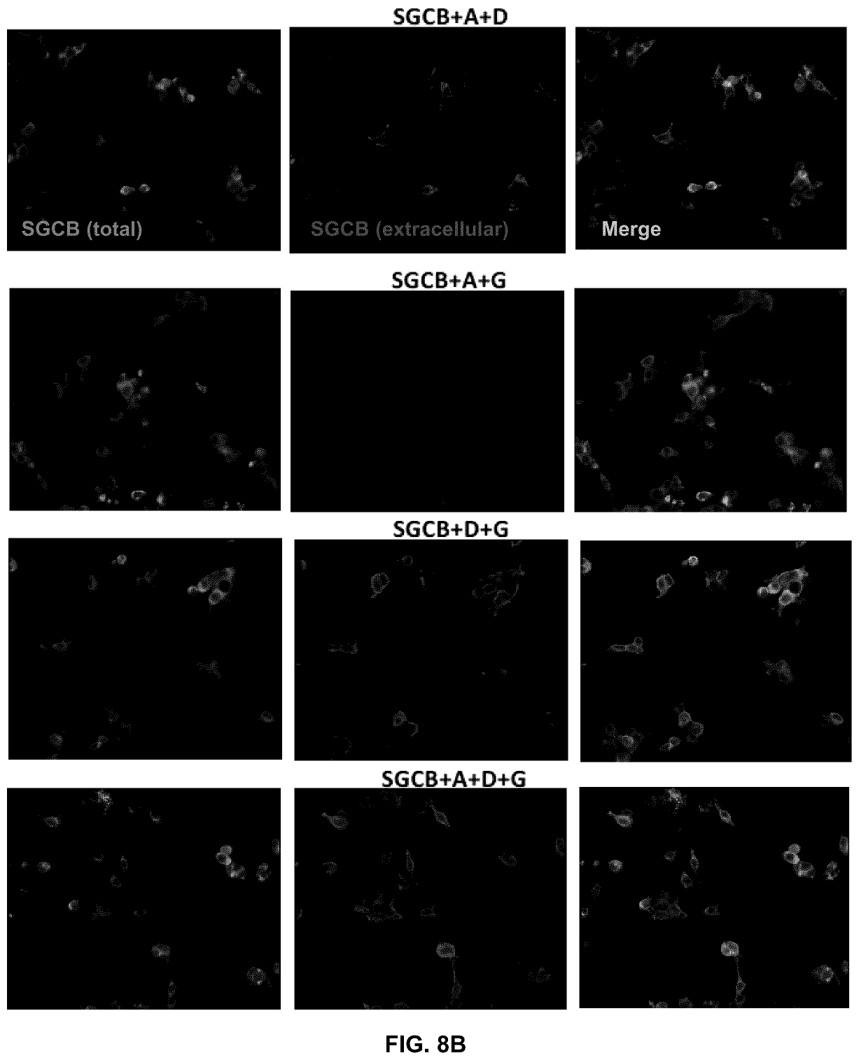

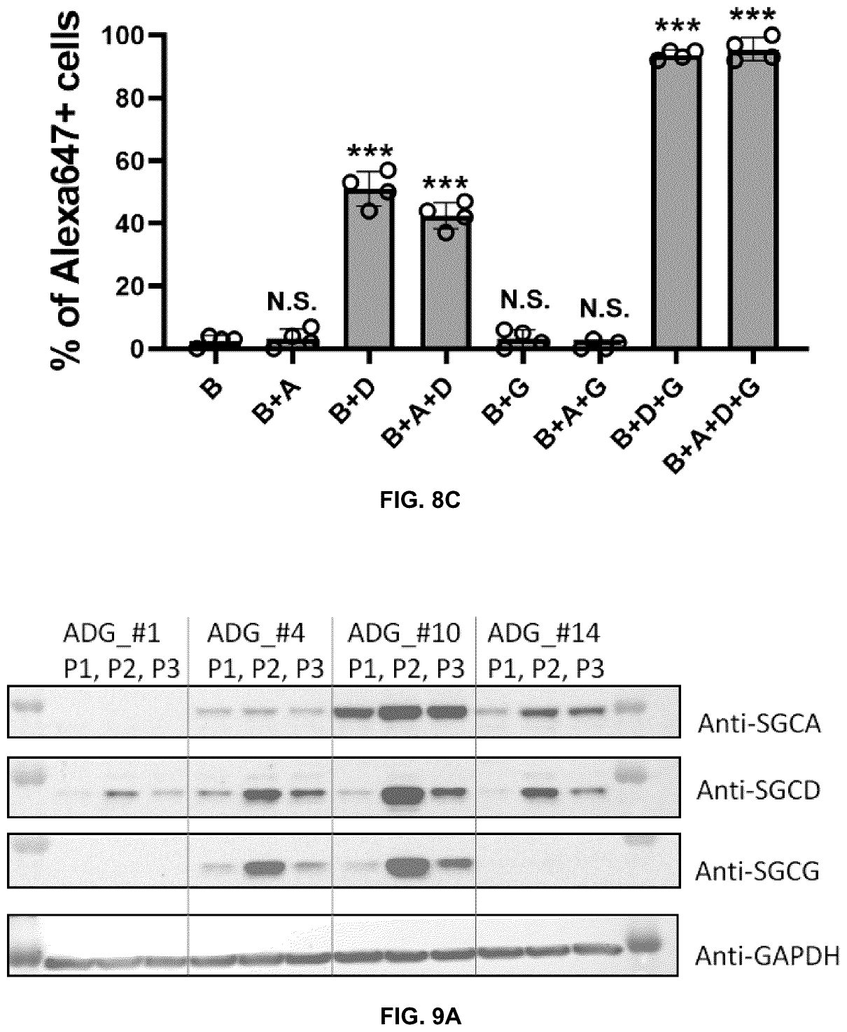

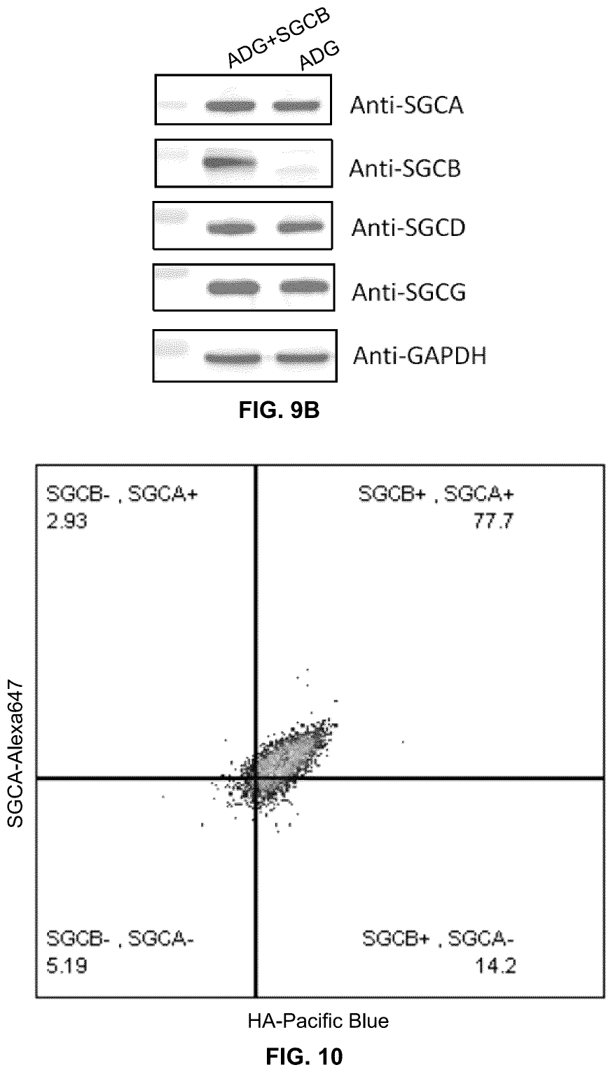

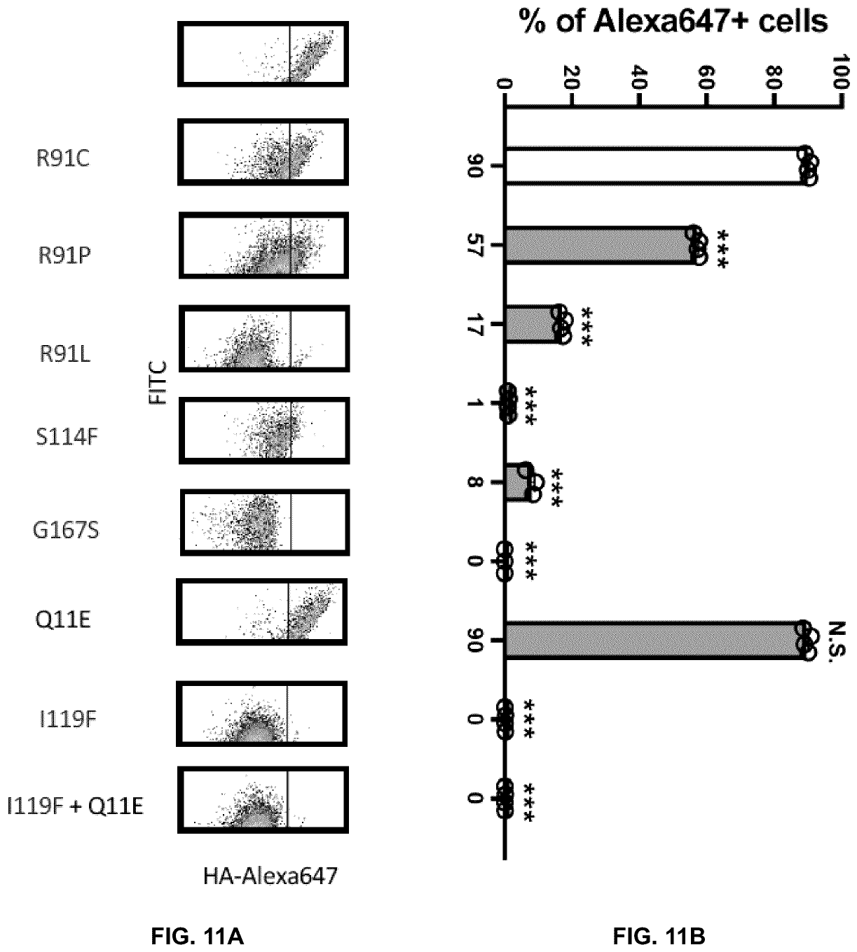

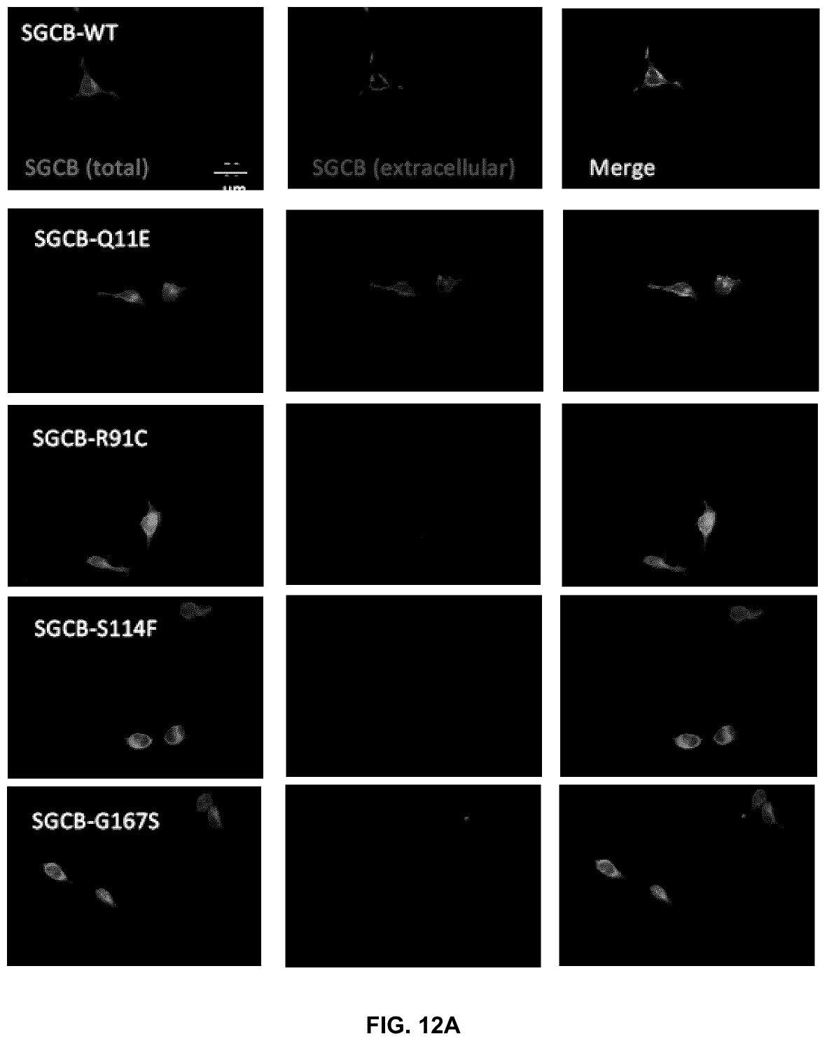

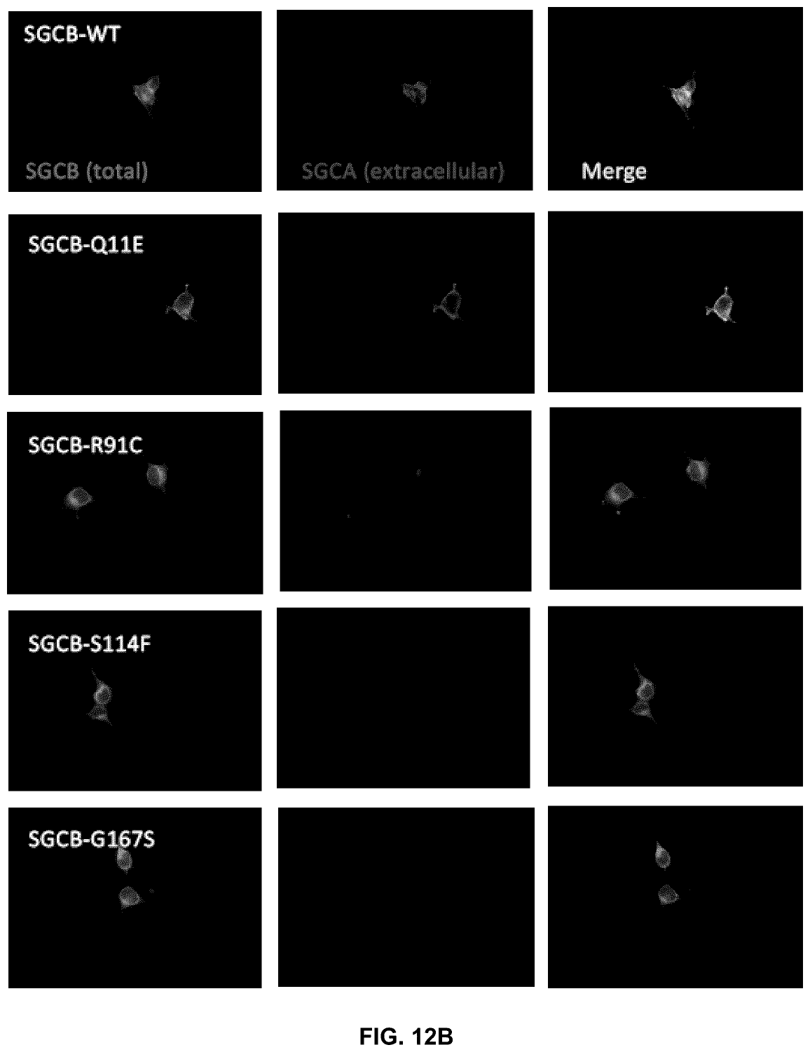

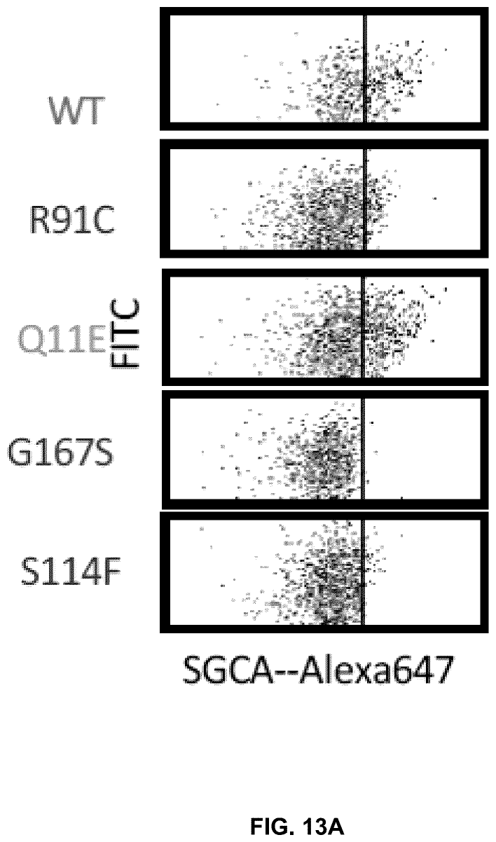

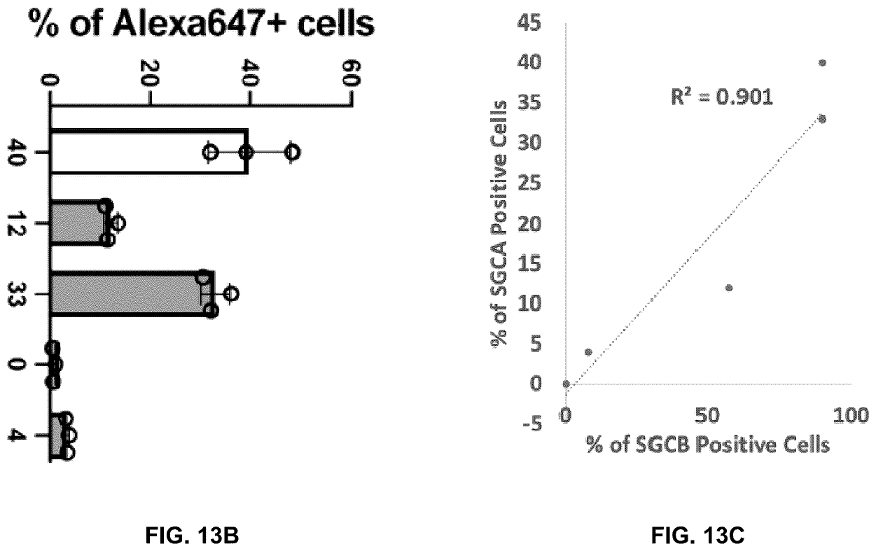

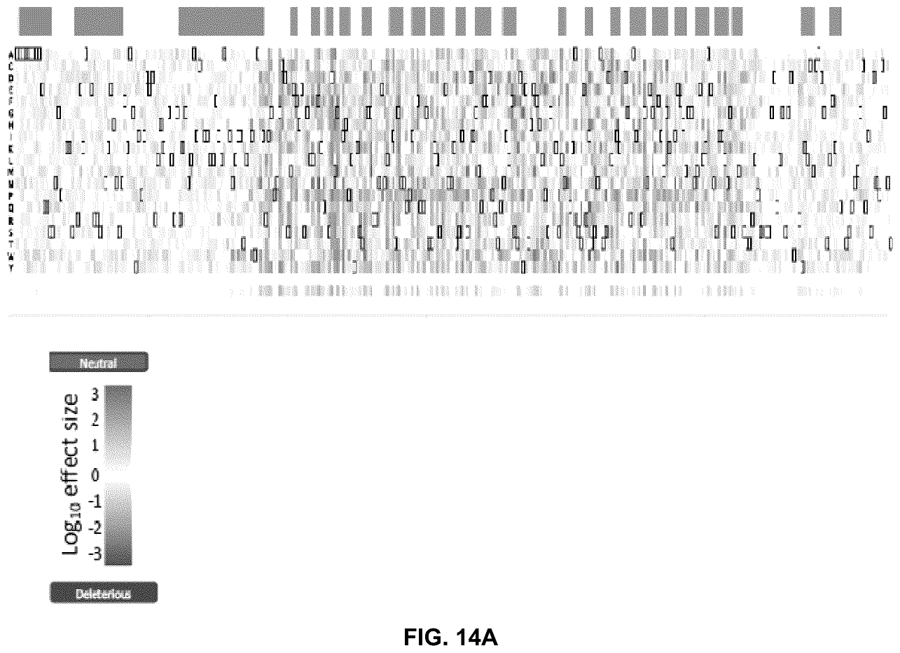

A human cell system was established to model SGCB variant function using engineered HEK293 cells. Testing for protein expression for the 4 sarcoglycan (SGC) proteins verified that HEK293 cells lacked detectable protein expression of any of SGC gene. Furthermore, expression of a SGCB fusion protein (YFP-SGCB-HA) alone led to minimal cell surface expression, as measured by immunofluorescence with an anti-HA antibody (FIG. 8A, FIG. 8B). Co-expression with the other 3 untagged SGC proteins (SGCA, SGCG, and SGCD), however, led to robust cell surface expression of WT YFP-SGCB-HA. To create a stable HEK293 cell line capable of reliably transporting SGCB to the cell surface, HEK293 cells were transduced with a mixture of lentiviruses designed to express SGCA, SGCD, and SGCG. Single clones from these transduced cells were isolated and tested at multiple passages for expression of the 3 stably expressing SGC proteins (FIG. 9A). One clone that expressed each SGC protein at similar levels was chosen and deemed ADG-HEK cells; it was used for all subsequent experiments (FIG. 9B). To preliminarily validate the assay, single missense mutations were introduced into YFP-SGCB-HA that are predicted to affect the cellular expression of SGCB because they have been established previously as pathogenic (G167S, S114F). In addition, one missense mutation (Q11E) was generated that has been reported as a VUS. Lentiviral expression of YFP-SGCB-HA-WT in ADG-HEK cells displayed strong cell surface expression in nearly all YFP-positive transduced cells using an HA antibody on unpermeabilized cells (FIG. 10). In contrast, ADG-HEK cells transduced with presumptive pathogenic variants had a significant decrease in cell surface expression of SGCB, as visualized by immunofluorescence of unpermeabilized cells (FIG. 11A, FIG. 11B, FIG. 12A, FIG. 12B). In contrast, the VUS YFP-SGCB-HA-Q11E had normal cell expression that was comparable to that of YFP-SGCB-HA-WT in ADG-HEK cells, as visualized by immunofluorescence of unpermeabilized cells, suggesting that this variant has no effect on its function. To create a high-throughput and quantitative assay for SGCB membrane expression, flow cytometry was performed on similar populations of cells as above. In addition, to explore whether an impairment in SGCB would lead to a secondary loss in SGCA cell surface expression, ADG cells were immunostained as transfected with YFP-SGCB-HA-WT or mutation-containing plasmids and unpermeabilized cells were immunostained with an antibody against the extracellular domain of SGCA. Consistent with a loss of SGCB cell surface expression, there was a secondary loss of extracellular SGCA cell surface immunofluorescence (FIG. 12A, FIG. 12B, FIG. 13A, FIG. 13B).

### A Functional Effect Map of SGCB Missense Variants.

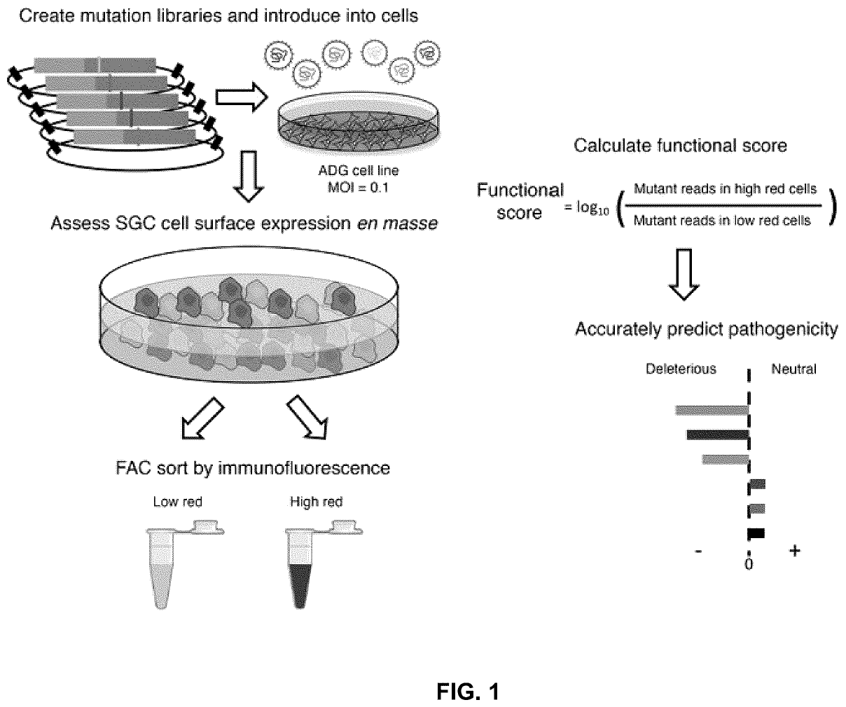

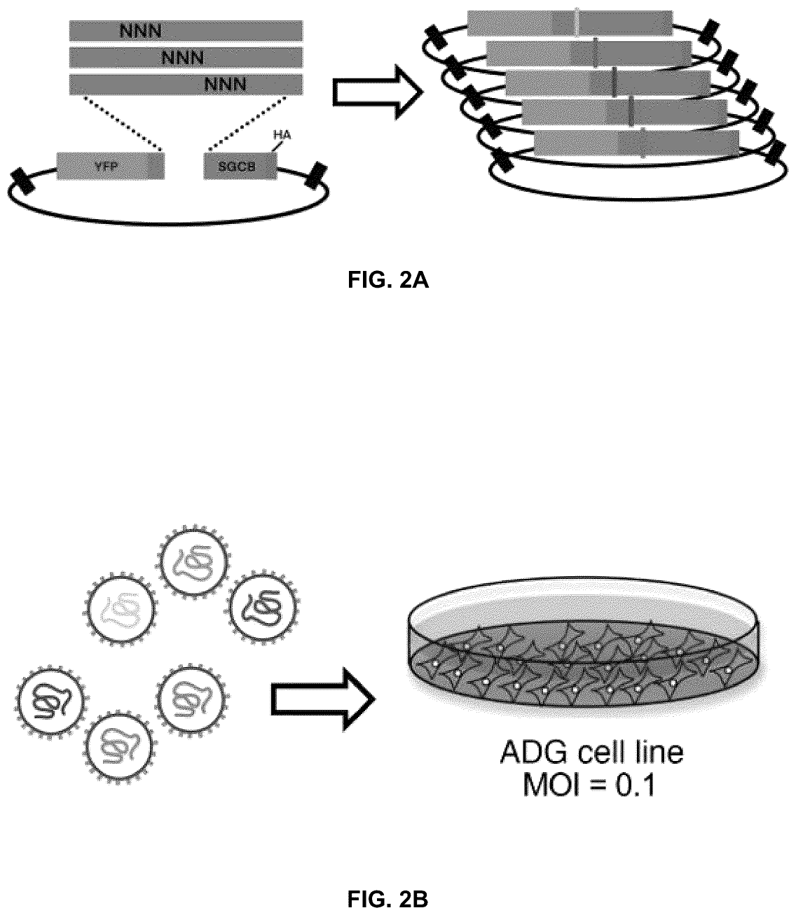

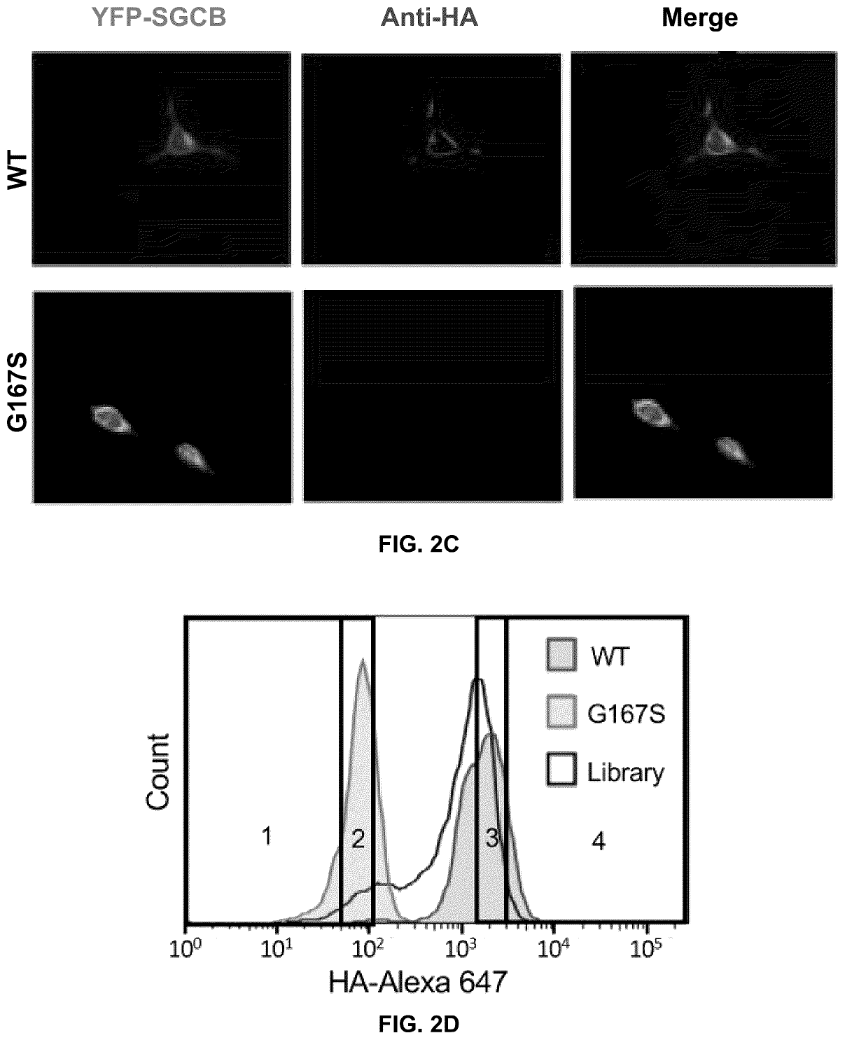

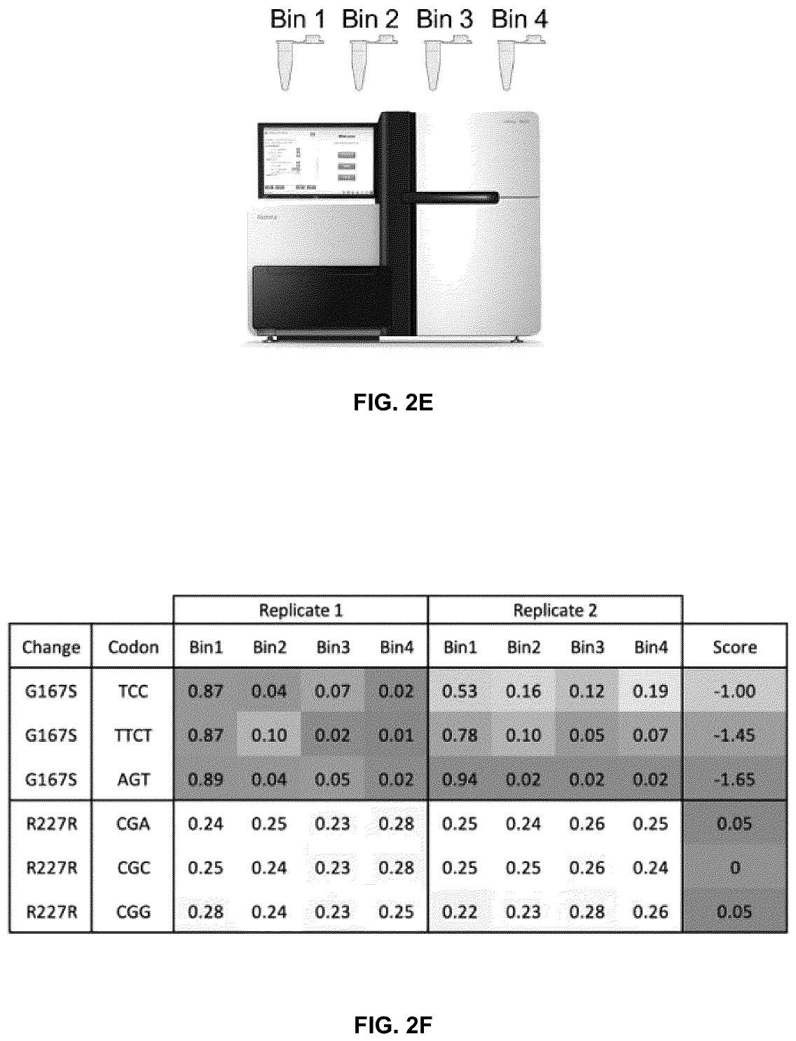

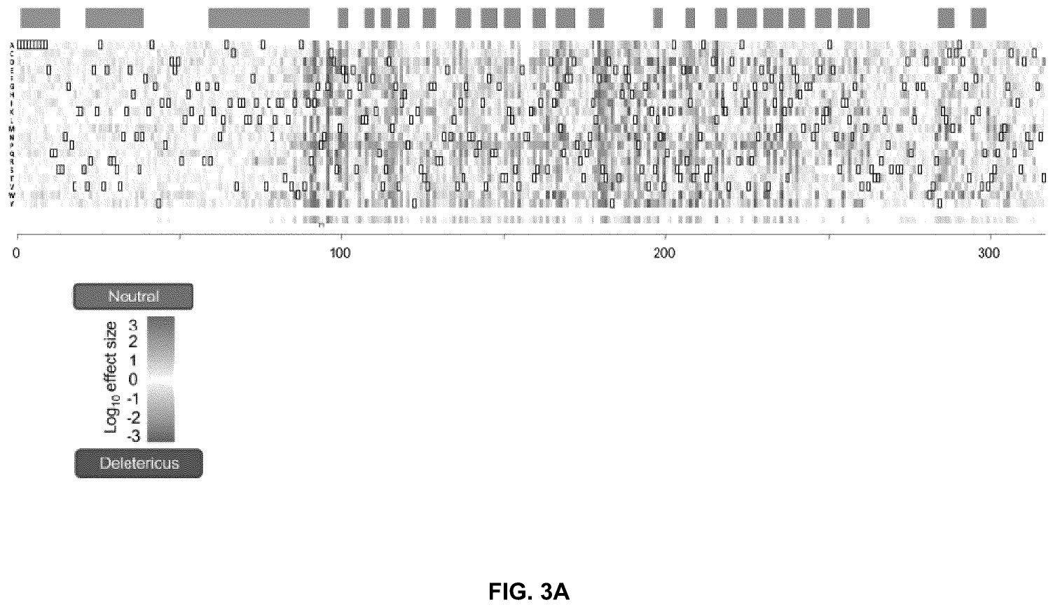

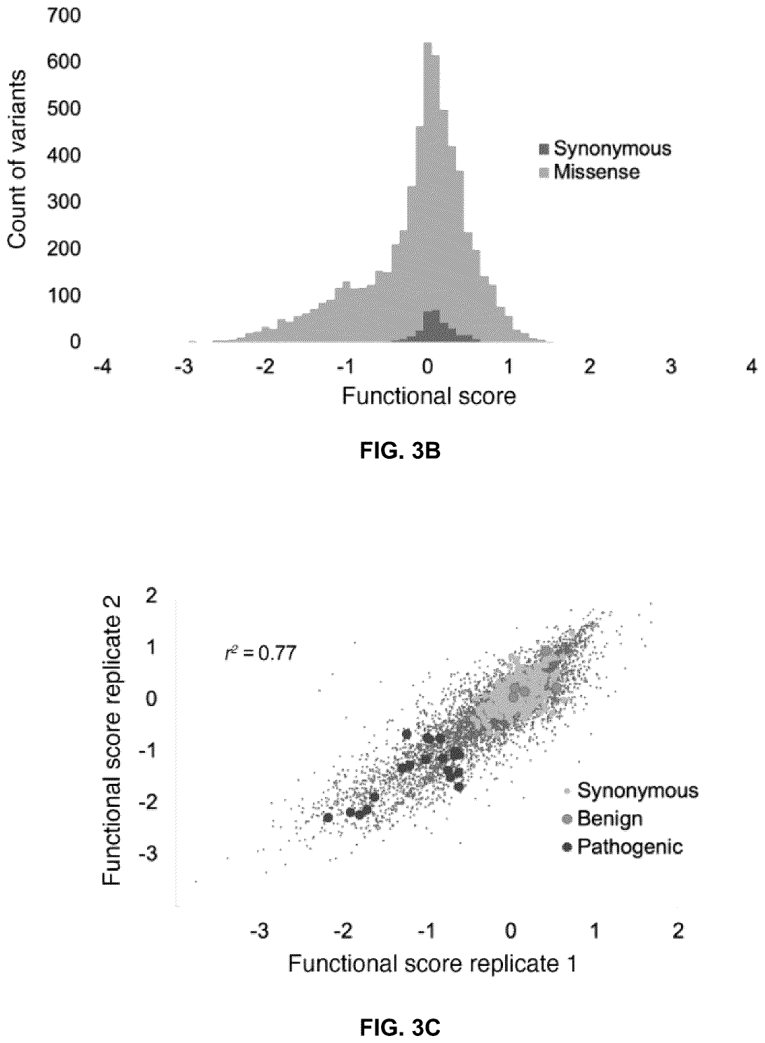

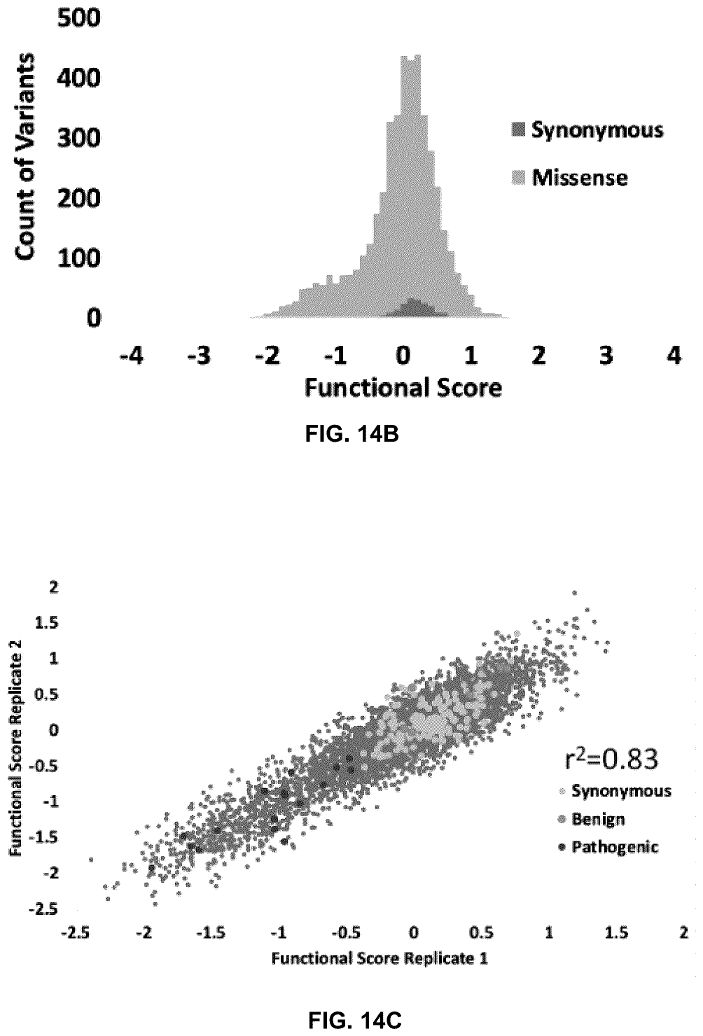

To test the effect of all possible missense SGCB variants, single amino acid saturation mutagenesis was used to generate libraries comprising every possible missense, synonymous, and nonsense variant. The SGCB cDNA (954 bp) was divided into 6 overlapping sub-libraries (204-225 bp) to allow full-length sequencing of each. The mutant cDNA library for each sub-library was cloned into the WT lentiviral YFP-SGCB-HA vector. ADG-HEK cells were transduced at low multiplicity (<0.1) to yield a population in which each cell expressed either 0 or 1 SGCB variant, recapitulating a hemizygous-like state in which to test the effect of each variant. Sequencing of both plasmid libraries and integrated libraries from genomic DNA demonstrated that nearly all single-codon mutations (98%) and nearly all amino acid changes (99%) were present and transduced into cells and the average depth of each mutation was generally uniform (range=50-1,000 reads per million). Each cell population transduced with a SGCB mutant lentivirus pool was first bulk-sorted for expression of YFP to select cells expressing SGCB. YFP-positive cells were then grown for an additional 5-7 days and split into 2 equal populations and stained either for HA to detect YFP-SGCB-HA surface expression or with an antibody against the extracellular domain of SGCA to detect cell surface of SGCA. Cells were sorted into 4 bins corresponding to the top and bottom 10% of cells for either YFP-SGCB-HA or SGCA cell surface expression. To determine the relative abundance of each mutation in each bin, amplicon sequencing was performed of each mutated sub-library. Functional scores for each variant were calculated for YFP-SGCB-HA cell surface expression (bin 1 vs. bin 4) and SGCA cell surface expression (bin 1 vs. bin 4) as the log 10 ratio of the variant's frequency in bin 4 (high expression) divided by its frequency in bin 1 (low expression), such that deleterious variants should score negatively and neutral variants positively (FIG. 1, FIG. 3B, FIG. 14B). Functional scores ranged from −2.9 to 1.46, with all synonymous variants having a score of more than −0.5 (average=0, range=−0.05 to 1.2). In contrast, nonsense mutations had an average functional score of −1.5±0.8. Using these values, cutoff scores were established for putative benign and pathogenic variants and normalized scores across chunks. Scores were not correlated with chunk of origin but did strongly reflect the protein domains and structural features predicted using AlphaFold2 (FIG. 3A). Generally, positions before amino acid position 85 show a reduced likelihood of pathogenicity, and amino acid positions within the extracellular domains of the protein, particularly those within predicted β sheets showed an increased likelihood of pathogenic functional scores and scores demonstrated a bimodal distribution (FIG. 3B). Functional scores were correlated across biological replicates (different transduced cell populations) (pair-wise Pearson's r2=0.77, FIG. 3C).

### Functional Scores Validate Existing Variant Interpretations.

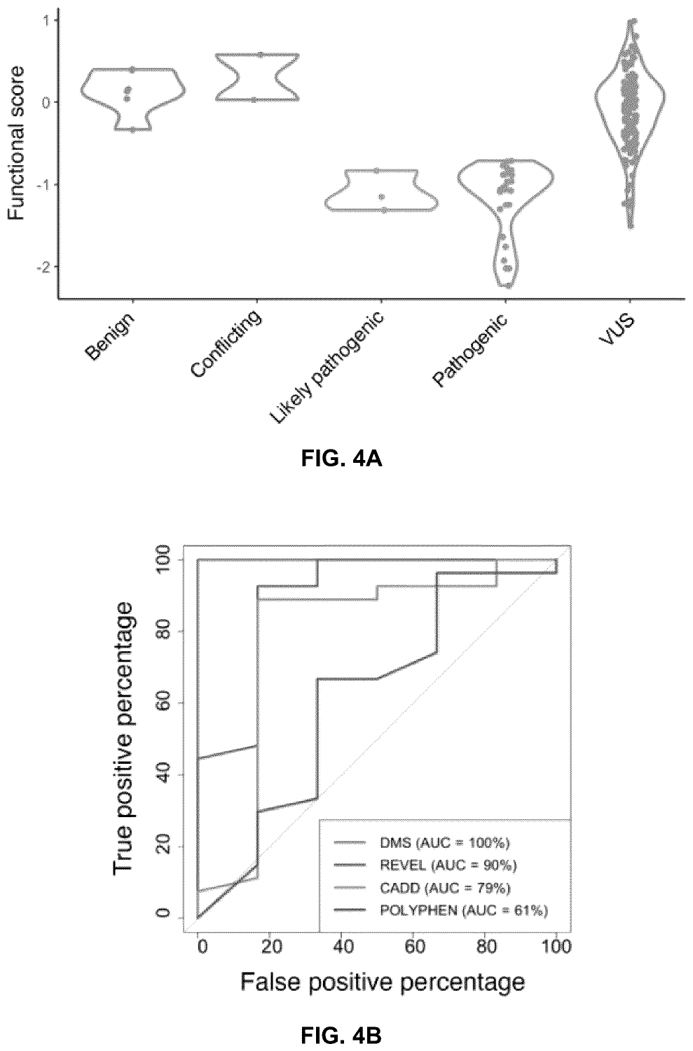

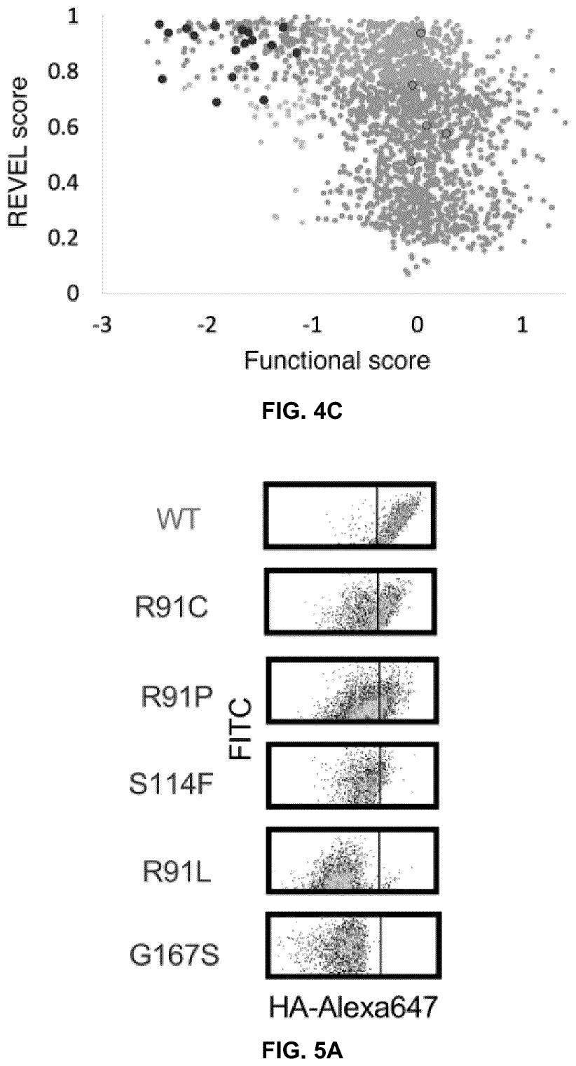

Having established cutoff scores, it was found that pooled measurements recapitulated existing variant interpretations from ClinVar and the Leiden databases and recapitulated scores derived from single variant experiments (FIG. 11A, FIG. 11B, FIG. 12A, FIG. 12B). Notably, functional scores agreed with the reported pathogenicity of all variants reported as pathogenic, likely pathogenic, benign or likely benign (FIG. 4A), corresponding to classification sensitivity and specificity of 100%. These data provide evidence for the interpretation of the 99 VUSs in ClinVar or the Leiden SGCB databases. Using the functional scores derived here, 12 of these (12%) unresolved variants are predicted as functionally deleterious (Table 1), a rate similar to the overall rate of nonfunctional variants possible from single nucleotide changes (283 of 2,250, 13%; P>0.05, 2-sided binomial test). The functional scores were also investigated of variants present in healthy populations. These databases were used to test the false positive rate of the functional assay. Presuming that no one in these cohorts had LGMD, it was expected that there would be no homozygotes with nonfunctional alleles by the assay and no compound heterozygotes with 2 nonfunctional alleles by the assay in any of these populations. First, the UK Biobank (UKBB) database was utilized. Among the UKBB population, there were 1,654 individuals with more than 0 nonsynonymous variants in SGCB. Of these, only 6 individuals harbored 2 nonsynonymous variants. In each instance, at least 1 of the 2 variants scored as functionally neutral/benign in the functional assay. It was therefore predicted that none of the 488,248 patients present in the UKBB exome sequencing cohort had SGCB-deficient LGMD. Next, the gnomAD database (version 2.1.1) lists 198 SGCB missense variants, all rare (maximum MAF, <0.2%). Of these variants, 20 variants (102 of 129,186, cMAF=0.00079) were deleterious in the assay (score, <˜0.5) and present in non-Finnish Europeans, with 0 of these observed as homozygotes. The most frequent variant that scored as pathogenic (S114F) is a known pathogenic variant in ClinVar and was observed in 68 non-Finnish European individuals in gnomAD, with none observed as homozygotes. Three SGCB missense variants were observed in the homozygous state in at least one population in gnomAD (R267C, F180L, and Y123S), but each scored as functionally neutral in the assay. These results reflect the mild negative selection on pathogenic variants in recessive disease genes that allows genetic drift to increase the frequency of even disease-causing variants below the level that would frequently produce homozygotes. Furthermore, the collective frequency of missense variants predicted were pathogenic in these 2 databases suggests a carrier frequency around 1 in 1,250-2,400 and, therefore, a population prevalence of SGCB-deficient LGMD of approximately 0.2-0.6 per million, ignoring the de novo mutation rate for this gene, which is consistent with various disease prevalence estimates.

### Functional Scores Outperform Bioinformatic Predictors.

Bioinformatic tools are often used to aid in the interpretation of clinical variants. Methods are seldom protein specific and are based largely on evolutionary conservation, which for highly conserved genes often lead to inflated sensitivity but limited specificity in pathogenicity predictions, the classification performance of functional scores was compared with those of 3 computational predictors: PolyPhen2, CADD, and REVEL. ClinVar pathogenic, ClinVar benign, and variants were used for which homozygotes have been observed in gnomAD or the UKBB (presumed benign) as a set of true positive and true negative variants. Measured functional scores were perfectly concordant with published pathogenicity assessments FIG. 4A). Functional scores outperformed each bioinformatic method in predicting pathogenicity, producing an AUC of 1; the next best predictor was REVEL, with an AUC of 0.9 (AUC range, 0.6-0.9; FIG. 4B). However, the value of REVEL scores in predicting pathogenicity is likely inflated, owing to the lack of known benign variants with which to evaluate it. Given the large number of variants with functional scores of more than 0 (i.e., functionally benign) but damaging REVEL scores (>0.75; 537 of 1,870; 29; FIG. 4C) remains possible that the false positive rate for REVEL scores (i.e., benign variants called pathogenic by REVEL) is substantially higher than estimated with the currently available set of ClinVar and Leiden variants. An additional caveat to the predictive value of bioinformatic tools is that the determined pathogenicity of variants present in ClinVar and other clinical databases is often partially based on these tools, making the probability of a “pathogenic” score from these tools higher than by chance in many cases.

Functional Scores Correlate with Disease Severity.

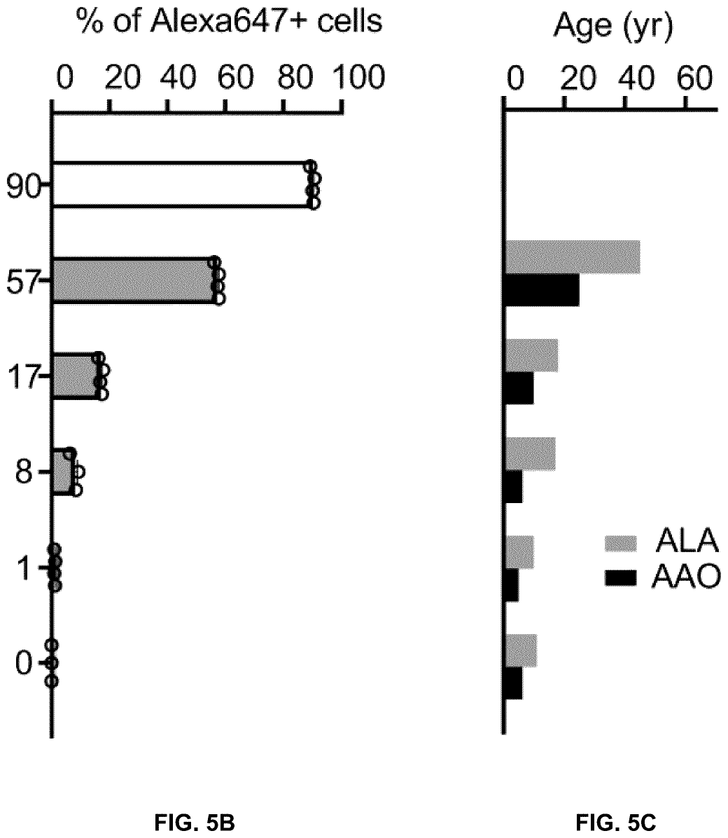

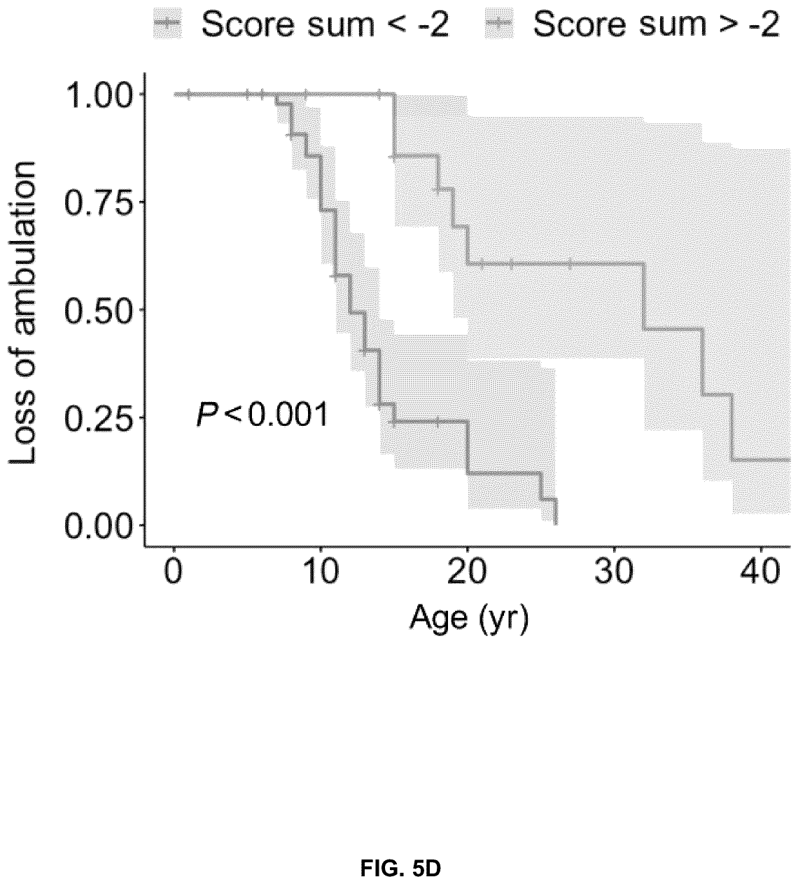

There is a wide range of phenotypes for patients with LGMDR4/2E; age at symptom onset ranges from less than 1 to more than 20 years of age, some patients require a wheelchair at as early as 7 years of age, and some patients never require a wheelchair. This phenotypic variability is most strikingly exemplified with missense variants residing at position arginine 91 in SGCB. Three variants have been reported at this residue, with patients presenting with a mild phenotype if homozygous for an R91C, an intermediate phenotype if homozygous for R91P, and a more severe phenotype if homozygous for R91L variant. To validate this finding using the functional scores, individual lentiviral constructs were generated expressing YFP-SGCB-HA with the R91C, R91P, and R91L variants. The R91C, R91P, and R91L variants had diminished expression of 57%, 17%, and 1%, respectively FIG. 5A, FIG. 5B), values that corresponded to the average age at onset and age at loss of ambulation for patients with these variants (FIG. 5C). It was further determined if the measured functional score could be used as a measure of disease severity more generally and to predict either age at onset or age at loss of ambulation (i.e., age at which the patient required a wheelchair) using all available clinical data for patients with missense variants. A Cox's proportional hazard analysis was performed comparing the age at onset or age at loss of ambulation among patients with 2 severely nonfunctional missense SGCB alleles (functional score sum, <˜2) with those with 2 fewer severe missense SGCB alleles (functional score sum, >−2). While there was no significant difference in the age at onset between these two groups, the age at loss of ambulation was significantly lower among patients with 2 severely nonfunctional SGCB alleles (Cox's proportional hazard, P<0.001) (FIG. 5D), and there was a significant correlation between age at loss of ambulation and functional score sum (r2=0.22, P=0.002), suggesting that the functional score derived here can not only predict pathogenicity but also disease severity.

### Functional Constraint Highlights Structural Features and Protein-Protein Interactions.

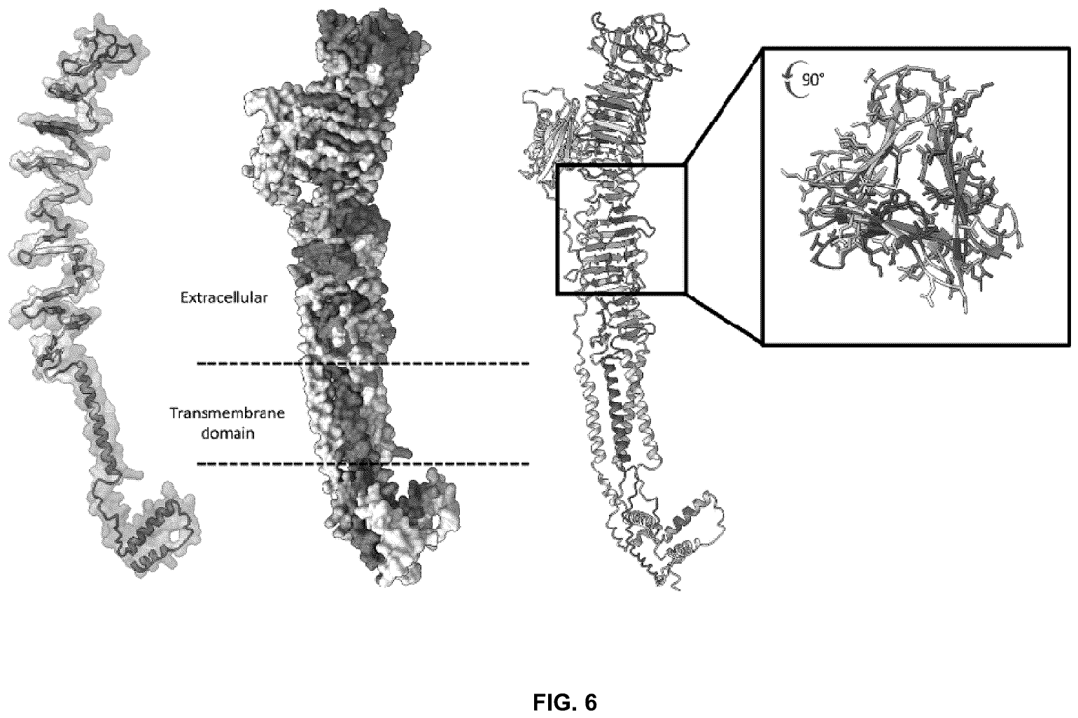

High-throughput functional screens, particularly those that test all possible amino acid changes, have the ability to inform knowledge of the structure-function relationships present within a protein or protein complex. For example, as expected, amino acid changes that resulted in conservative changes (i.e., acidic to acidic amino acids) produced deleterious functional scores less often (101 of 1,171, 8.6%) than those that resulted in nonconservative changes (600 of 3,482, 17.2%; P<4×10−6). A strong relationship between protein domain and functional score also was revealed when comparing the number of non-functional amino acid changes (functional score, <−0.5) in the cytoplasmic (54 of 1,282, 4.2%) or transmembrane domain (13 of 351, 3.7%) of SGCB with those in the extracellular topological domains of the protein (637 of 3,020, 21.1%; P=7.6×10−34). Because the SGCB protein has not yet been crystalized, no structural models are known for SGCB or the sarcoglycan protein complex. In order to further determine the relationship between functional scores and protein structure, a model of SGCB protein structure was produced using the multimer function of AlphaFold2 in the presence of SGCA, SGCD, and SGCG. The model produced was substantially different than the AlphaFold2 model produced using SGCB alone and revealed a structure in which SGCB, SGCD, and SGCG form a triple-helical quaternary protein structure with cobinding β sheets forming an interprotein β-barrel-like structure (FIG. 6). It was observed that the predicted structures form repeating regions of mutational intolerance corresponding to β sheet amino acids with inward-facing side chains harboring an excess of deleterious amino acid changes compared with outward-facing B sheet amino acids. Accordingly, the average number of interprotein contacts (<4 Å) among amino acid changes with functional scores of less than 0 was significantly greater than that with functional scores more than 0 for SGCB-SGCD (1.9 vs. 1.34, P<2.4×10−8) and SGCB-SGCG (1.97 vs. 1.65, P<9.3×10−4) interacting surfaces, the 3 proteins forming the triple-helical protein structure, and fewer for SGCB-SGCA interacting surfaces (0.28 vs. 0.71, P<8.7×10−4). The proportion of sites with at least one interprotein contact between SGCB and either SGCD or SGCG for amino acid positions with reported pathogenic variants in SGCB was also significantly greater than that of sites without known pathogenic variants (100% vs. 60%, P=3.7×10−4), further supporting the claim that intermolecular interactions are critical for SGCB function. Similar to previous deep mutational scans, amino acid changes resulting in proline were also significantly enriched for deleterious functional scores compared with other amino acid changes (40% vs. 24%, with scores <−0.5, P=3.9×10-14). One region with strong mutational intolerance but unclear function is amino acids 90-99. Of particular note is position R91, which harbors 3 different known pathogenic variants (R91C, R91L, R91P). It may be that these positions on the cusp of the transmembrane domain (AA 60-90) are important for proper orientation of the protein complex in the membrane or are critical for initiating the helical structure of the 3 core SGC genes within the larger complex. It is unclear, however, why position R91 harbors more observed pathogenic variants than other sites in the SGCB protein.

### Inter-SGC Protein Interactions Accurately Predict SGCD and SGCG Pathogenic Mutations.

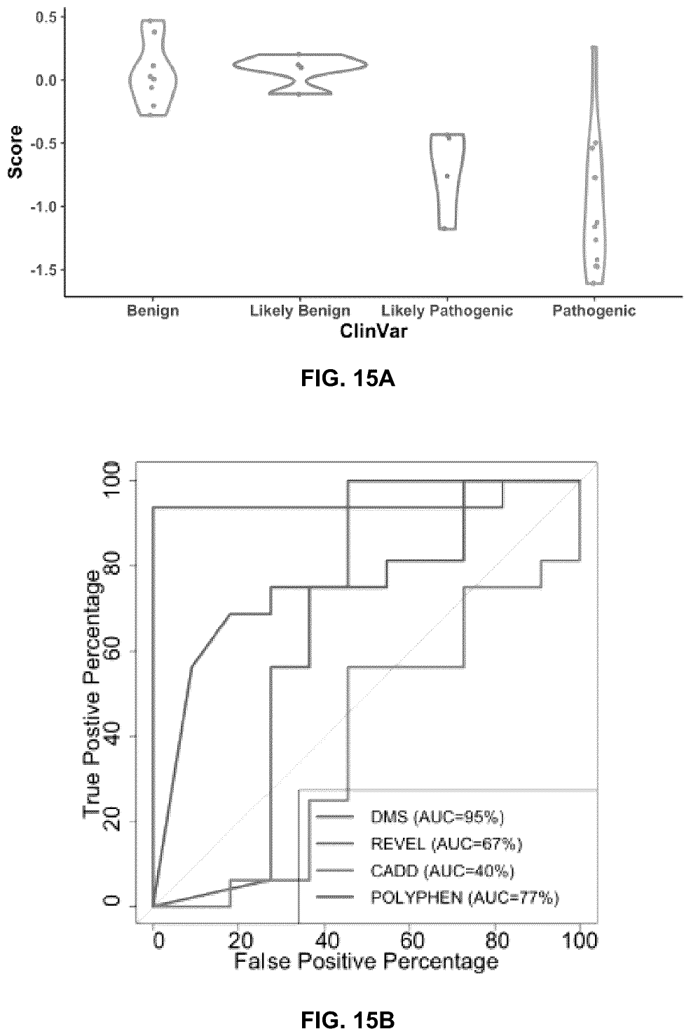

Because of the significant enrichment of damaging mutations at amino acids in SGCB that physically interact with SGCD and SGCG in the AlphaFold2 multimer protein structure model, analogous changes at residues in SGCG and SGCD that interact with constrained SGCB residues also lead to pathogenic changes. To support this, the protein sequence of SGCB was aligned to that of either SGCG or SGCD using Clustal Omega and it was found that a helices and B sheets predicted by AlphaFold2 nearly perfectly aligned between SGCB and both SGCG and SGCD and that SGCD and SGCG are highly similar, with 53% of their amino acids being identical. Functional scores were superimposed from SGCB onto the aligned amino acids in either SGCG or SGCD (i.e., L194S in SGCG corresponds with 1218S in SGCB) determined from these protein alignments and found that nearly all clinically determined pathogenic variants have corresponding pathogenic scores in SGCB (range=−1.61 to 0.26) and all clinically determined benign variants have corresponding benign scores in SGCB (range=−0.46 to 0.47; FIG. 15A, FIG. 15B). Furthermore, pathogenic variants in SGCG or SGCD almost exclusively reside in B sheets (14 of 16 variants) involved in SGC-SGC contacts whereas nearly all benign variants reside within the intracellular or transmembrane domains of the proteins or in the extracellular domains of the proteins but outside of a β sheet (7 of 10 variants). These predictions of pathogenicity outperformed CADD, REVEL, and Polyphen scores (DMS AUC=0.95, FIG. 15A, FIG. 15B) in predicting pathogenicity of known variants in these genes. Together these findings suggest that DMS combined with structural information, either determined empirically or in silico, can inform pathogenicity predictions across proteins with similar structure and function.

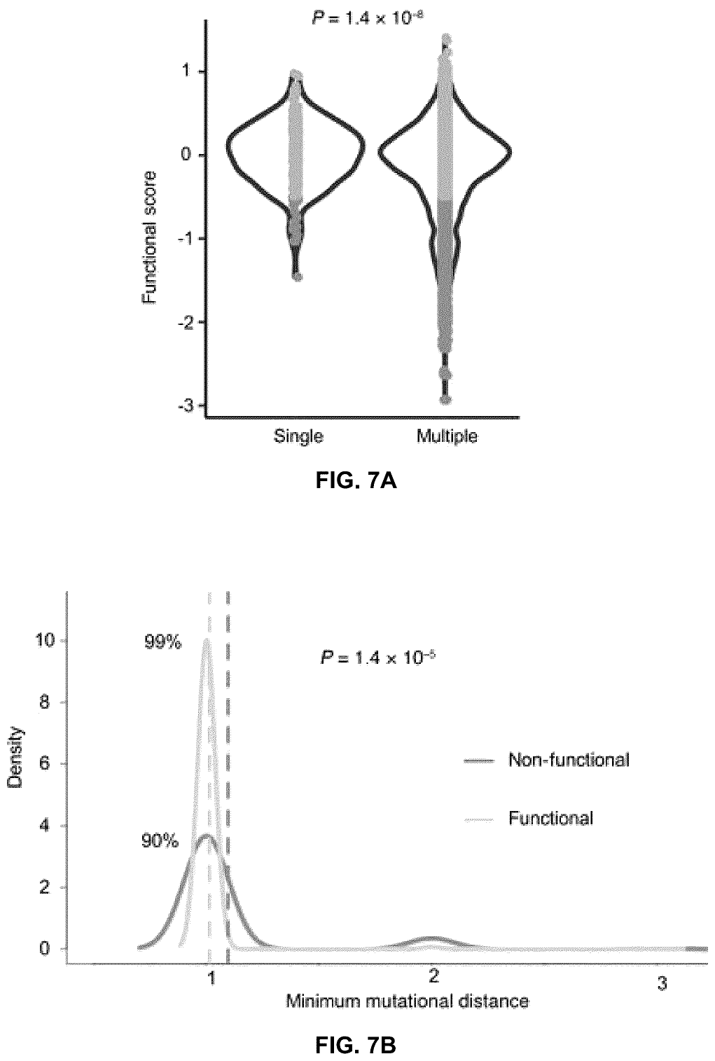

Insights into codon evolution from single amino acid saturation mutagenesis. Unlike single nucleotide mutagenesis, which enables the measurement of all possible variants generally accessible during evolution, single amino acid saturation mutagenesis enables measurement of amino acid changes not easily accessible by evolution, i.e., codon changes requiring 2-3 nucleotide changes. It was determined whether amino acid changes that resulted from single nucleotide changes were more or less likely to lead to a nonfunctional protein compared with amino acid changes that are only possible following 2-3 adjacent nucleotide changes, consistent with the well-established finding that more similar codons tend to encode more biochemically similar amino acids. While no difference was observed between the average functional score of amino acid changes caused by single nucleotide changes and those same amino acid changes resulting from multinucleotide changes (t test, P>0.05), a strong enrichment of deleterious functional scores was observed among amino acid changes only possible from multinucleotide changes compared with amino acid changes possible from single nucleotide changes (score, −0.18 vs. 0.01, t test, P=1.4×10⁻⁸; FIG. 7A) and the average mutational distance of deleterious amino acid changes was significantly greater than that of neutral amino acid changes (2.4 vs. 2.2 nucleotide changes, P=1×10⁻⁵⁸; FIG. 7B). This observation was also true in a recently published DMS of MSH2, where 7% of amino acid changes reachable by a single nucleotide change resulted in a nonfunctional protein (MSH2 functional score, >0) compared with 12% of amino acid changes possible only by more than 1 nucleotide change that resulted in nonfunctional protein (t test, P<5×10−21). To test more specifically that specific codons were selected by evolution to be maximally distant from deleterious amino acid changes, the average distance was calculated of possible codons at each position that encode the WT amino acid and compared it with the observed distance of the codon chosen by evolution. The difference between the utilized codon and the average of possible codons for functionally deleterious amino acid changes was significantly greater than for those that were functionally neutral (+0.029 mutational distance, paired t test, P=8×10−6), whereas there was no significant difference for neutral amino acid changes (−0.004 mutational distance, paired t test, P=0.26). Finally, when restricting the analysis to only those amino acid changes possible by 1 nucleotide change, deleterious amino acid changes were more often encoded by a codon that requires a greater number of nucleotide changes to obtain the amino acid change compared with neutral amino acid changes (82% vs. 70%, P=1.4×10−13), effectively maximizing the mutational distance from the current amino acid to the array of possible deleterious amino acids by codon choice.

### Discussion

The sarcoglycan genes and SGCB in particular are among the most frequently mutated genes underlying LGMD. Like many recessive disease genes, affected patients carrying biallelic loss-of-function variants (i.e., premature termination codons or indels resulting in frame shifts) provide strong evidence for variant pathogenicity. However, this evidence is lacking for the majority of missense variants observed in patients. Immunofluorescence based cellular assays of sarcoglycan function provide the necessary evidence (ACMG criteria PS3) to aid in the interpretation of observed and yet unobserved genetic variation. As described herein, massively parallel assays of SGCB function, SGCB cell surface expression and SGCA cell surface expression were performed and integrated, and a near-complete map was generated of the functional effect of missense variants in the LGMD gene SGCB.

Measured functional scores for variants are highly accurate in predicting the pathogenicity of known disease-causing variants, outperforming the newest prediction algorithms. This is the first time that a full-length muscular dystrophy gene has been subjected to DMS, suggesting that pooled functional screens are a viable method of large-scale functional assessment of protein-coding genetic variation in other muscular dystrophy genes (SGCA, SGCD, SGCG, etc.), particularly those whose cell surface expression is key to their function.

Nearly all known pathogenic variants in SGCB that have been evaluated in primary tissue samples from patients show strong effects on the cell surface expression of not only SGCB but other members of the dystrophin-associated protein complex, particularly the other 3 sarcoglycan proteins, SGCA, SGCD, and SGCG. Accordingly, the functional measurements were highly consistent with expert-reviewed variant classification records from the ClinVar database and the Leiden genetic variant database that often use sarcolemmal expression in patient muscle tissue as their functional evidence. At least one goal is to provide functionally relevant evidence to be used in variant resolution, which would satisfy the requirements set forth by the ACMG to add strong evidence for the pathogenicity of variants with negative functional scores in the assays (PS3 criteria). Although the functional scores defined here were perfectly accurate in predicting the pathogenicity of variants with strong evidence in ClinVar, one variant (E28G) was listed in the Leiden SGCB database as “benign” without any cited evidence. The assay results would suggest that this variant is mildly deleterious (functional score, −0.33) but not below the cutoff of −0.5 for classifying a variants as nonfunctional despite being in the less constrained cytoplasmic domain of SGCB. The functional effect maps disclosed herein, therefore, enable reclassification of variants with limited evidence present in clinical databases.

It was found that measured functional scores were a better predictor of known pathogenic and benign variants than Polyphen2, CADD, or REVEL scores. Although the predictive value of REVEL was high using the relatively small number of known pathogenic/benign variants, true false positive rate for REVEL was estimated to be substantially higher when a larger number of confirmed disease-causing variants are defined. If using conservative cutoffs for a benign function score of more than 0.5 and a deleterious REVEL score of more than 0.75, 420 of 1,870 or 22% of single-nucleotide variants would be considered false positives (i.e., deleterious by REVEL and benign by DMS score), whereas only 1 known benign variant lies within this range.

Our findings that disease severity, as measured by age at loss of ambulation, are related to functional score in the assay suggests that cell surface expression of SGCB and the SGC protein complex is potentially a quantitative trait that determines, in part, the stability of the sarcolemma and the integrity of muscle cells over the course of a lifetime. Functional scores as measured herein may be determined to correlate with more subtle muscle traits, such as sarcopenia or exercise intolerance in the general population, at least among individuals with protein-altering genetic variation in these genes.

The pattern of deleterious amino acid changes across the SGCB gene closely mirrored the predicted protein structure produced using AlphaFold2. The co-occurrence of deleterious amino acid changes within predicted β sheets highlighted the importance of interprotein interactions, particularly between SGCB and SGCD, in producing a functional protein complex capable of being assembled and transported to the cell membrane. Intriguingly, there was minimal effect when amino acids within the intracellular domain or transmembrane domain of SGCB were changed. Furthermore, the importance of amino acid 91 is not yet clear, although it may be that its position at the transition between the transmembrane domain and the extracellular domain is critical for some yet undetermined function. Overall, the scores and their pattern corroborate the predicted AlphaFold2 structure and improve the understanding of the domains and interactions important for SGCB function. This was further demonstrated by the ability to predict the pathogenicity of SGCD and SGCG variants with high accuracy using SGCB functional scores and knowledge of the structural relationship between the 3 proteins. By aligning the 3 genes' protein structures and superimposing SGCB scores on SGCD and SGCG, further insight was gained into the importance of interprotein contacts by accurately predicting pathogenic variants in these two related proteins. Only one known pathogenic variant in either SGCG or SGCD (C283Y in SGCG) was predicted to be benign as it aligned to SGCB position C307Y (functional score, 0.26). The site is within the C-terminus of the protein, not within a β sheet, and has limited contacts with other SGC proteins (4 contacts with SGCG and 0 with the other 2 SGC proteins). The mechanism of functional disruption of this variant, if it is truly pathogenic, remains unclear. There may be other proteins that the amino acid at this position interact with that affect SGCG function but do not alter SGCB or SGCA trafficking. However, direct functional screening of all variants in both SGCD and SGCG will be required to confirm these predictions.

Overall, SGCB is moderately tolerant of protein-altering genetic variation, with 16% of single-nucleotide missense variants demonstrating nonfunctional scores (score, <−0.5). This number jumps to 30% when considering all possible amino acid changes, however, implying that evolutionary forces lead to the selection of specific amino acids and even specific codons with fewer nonfunctional alleles reachable by single nucleotide changes. This is the first time this phenomenon has been reported in DMS data and that this helps to explain the relatively low rate of pathogenic variants observed in human populations despite the high degree of evolutionary conservation across species. These data suggest that proteins may have evolved a buffer against deleterious protein alterations by selecting amino acids and codons with a lower probability of producing nonfunctional proteins.

The bimodal distribution of functional scores is similar to previous deep-mutational scanning reports, with most SGCB missense variants demonstrating either a clear nonfunctional score or a neutral score. It is yet unknown, however, whether functional scores between the means of these functional classes represent true quantitative measures of protein function or experimental noise. For example, there are 13 VUSs with functional scores between those of the highest scoring known pathogenic variant (R91C, score, −0.72) and the lowest scoring known benign variant (E28G, score, −0.33). No known pathogenic or benign variants exist within these borderline functional scores; however, this may be due to sampling bias with only those patients with severe phenotypes and clear genetic findings entered into clinical databases. When more clinical data from patients with a wider range of disease severity are investigated in the context of these functional scores, individuals can be identified with more intermediate phenotypes and correspondingly intermediate functional scores. For variants that appear to act as hypomorphs in functional assays, it is unclear whether these are likely to be considered pathogenic in the strictest sense or rather should be considered factors contributing to a spectrum of limb-girdle muscle weakness present in the general population. The relationship observed between functional score and age at loss of ambulation, even among patients with 2 alleles in the pathogenic range of functional scores, suggests that SGCB function is not purely normal or loss of function but a quantitative trait captured at least in part by measuring its ability to complex with other SGC genes and be transported to the cell surface as the assay measures. As large data sets of nonsyndromic individuals are sequenced and integrated with clinical information and physiological measurements, it may be possible to correlate more directly the role of SGCB genetic variation in the range of normal muscle function in addition to disease risk as explored here.

As demonstrated herein, high-throughput functional assays can accurately measure the effect of protein-coding genetic variation in the LGMD gene SGCB. The disclosed map of functional effects has the ability to improve classification of variants observed in patients with LGMD and aid understanding of the structure of an important member of the dystrophin-associated protein complex. When used together with available lines of evidence, these results will add confidence to variant interpretation and potentially allow patients with pathogenic variants to be treated with gene-specific therapeutics for which they would have otherwise been ineligible. Additional methods of the present disclosure include additional SGC and muscular dystrophy genes with similar pathological mechanisms.

### Materials and Methods

### Plasmid Constructs.

The full-length cDNA of human WT SGCA, SGCD, and SGCG was synthesized and individually cloned into a lentiviral vector by Genewiz. A YFP-SGCB construct was also synthesized and cloned into the same lentiviral vector expressing WT human β-sarco-glycan followed by YFP. A HA tag was further added to the C-terminal end of the SGCB by site-directed insertion mutagenesis (Takara). The new construct was termed YFP-SGCB-HA. Single or double variants in β-sarcoglycan were introduced by site-directed mutagenesis according to the manufacturer's protocol (Takara). All constructs were verified by Sanger sequencing (Genewiz).

### Cell Culture and Generation of ADG Stable Cell Line.

HEK293 (ATCC, CRL-1573) were cultured in DMEM (high glucose, MilliporeSigma), supplemented with 10% fetal bovine serum (Atlanta Biologicals). Cells were maintained in a humidified atmosphere of 5% CO2 at 37° C. During routine passaging, cells were washed with PBS and dissociated with trypsin-EDTA 0.05%. HEK293 cells were tested for endogenous α-, β-, γ-, and δ-sarcoglycan by Western blot assay; all were undetectable. To generate monoclonal stable cell lines coexpressing α-, γ-, and δ-sarcoglycan, HEK293 cells were tri-transduced with lentiviruses containing WT SGCA, SGCD, and SGCG as described above. Colonies formed by expansion of single cells were screened by PCR and Western blot for the presence of human sarcoglycans (FIG. 9A). Clone 10, expressing α-, γ-, and δ-sarcoglycan at about 1:1:1 stoichiometry, was used in the described experiments and deemed the ADG-HEK cell line. Protein expression of all 4 SGC proteins was confirmed after transduction of ADG-HEK cells with WT SGCB lentivirus by Western blot (FIG. 9B).

### Western Blotting and Immunocytochemistry on Nonpermeabilized Cells.

Cells were lysed by the addition of RIPA buffer (50 mM Tris-HCl, pH 7.4, 150 mM NaCl, 1% NP-40, 0.25% Na-deoxycholate, and 1 mM EDTA) and protease inhibitor cocktail (MilliporeSigma). Protein concentrations of lysates were determined using the BCA Protein Assay Kit (Thermo Fisher Scientific). Equal amounts of protein were run on a 10% gel and transferred to a nitrocellulose membrane. The blots were blocked with 5% nonfat dry milk in PBS for 1 hour and probed with anti-α-sarcoglycan (Santa Cruz, sc-390647/F-7), β-sarcoglycan (Santa Cruz, sc-393679/F-6), γ-sarcoglycan (Leica, NCL-d-SARC/Ad1/20A6), and δ-sarcoglycan (Lei-ca, NCL-g-SARC/35DAG/21B5) at 4° C. overnight. Secondary antibodies included horseradish peroxidase-labeled anti-mouse IgG (Cell Signaling, 7076). Blots were developed with ECL (GE Healthcare). Images were taken using the G: Box Chemi XT4, Genesys (version 1.1.2.0, Syn-gene). For immunocytochemistry, cells were fixed using 4% paraformaldehyde but not permeabilized. These cells were then stained for cell sur-face expression using an anti-HA antibody (Biolegend, 682404/16B12) followed by staining with an Alexa Fluor 647 goat anti-mouse secondary antibody (Thermo Fisher Scientific, A-21236), an Alexa Fluor 647-conjugated anti-HA antibody (Biolegend, 682404/16B12), a Pacific Blue anti-HA antibody (Biolegend, 901525/16B12), or an Alexa Fluor 647-conjugated anti-α-sarcoglycan antibody (Santa Cruz, sc-390647 AF647/F-7). Images were taken using a fluorescence microscope (Nikon 80i).

### Mutation Library Construction.

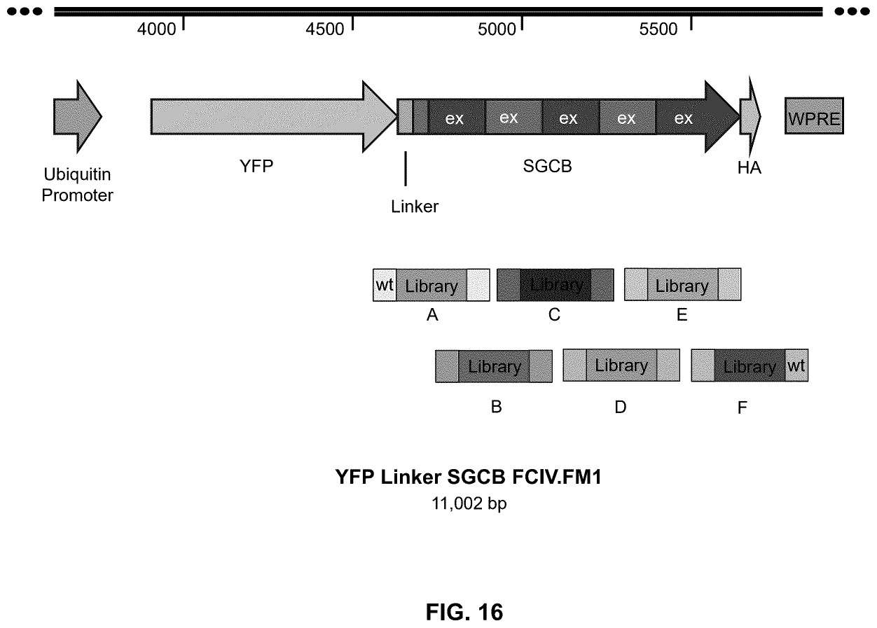

To achieve all possible amino acid substitutions of the entire SGCB (954 bp, 318 amino acid residues), the complete library was divided into 6 sublibraries (FIG. 16), and each contained a different 144-162 bp fragment of the SGCB gene (SGCB-A, residues 1-54; SGCB-B, 55-108; SGCB-C, 109-162; SGCB-D, 163-216; SGCB-E, 217-270; and SGCB-F, 271-318). Each sublibrary was custom synthesized by Integrated DNA Technologies as a pool of oligonucleotides such that each oligonucleotide encoded an “NNN” at one amino acid position but was otherwise homologous to the template. To enable initial PCR amplification and insertion of sublibraries into plasmids by Gibson assembly (described below), WT SGCB homologues sequences (>20 bp long) were added at both ends of the sublibraries.

For cloning of each sublibrary into lentiviral vector YFP-SGCB-HA (described above), the synthesized oligonucleotide pools were dissolved and diluted at 1:100 and used as template for PCR amplification with CloneAmp HiFi Polymerase (25 cycles, Takara). The product was purified using the Nucleospin Gel and PCR Clean-up kit (Takara). Backbone plasmid YFP-SGCB-HA was PCR amplified with primers that are complementary to the WT SGCB homologues sequences at the ends of each sublibrary. The resulting linearized backbone that carried the remaining template sequence was digested with DpnI to deplete starting plasmid, separated from nonspecific fragments by electrophoresis on a 1% agarose gel, and purified using the gel clean-up kit (Nucleospin). Each sublibrary was inserted into YFP-SGCB-HA plasmids via Gibson assembly (NEBuilder HiFi DNA assembly Master Mix) according to the manufacture's protocol, so that it replaces the equivalent part of the WT SGCB cDNA sequence. The assembly reactions were purified and transformed into E. cloni DUO Electrocompetent cells (Lucigen) with 5% of cells plated to estimate cloning efficiency and 95% of cells grown in liquid media for 12-16 hours at 37° C. in LB supplemented with 100 mg/mL ampicillin. An estimated >40,000 colonies per section was obtained to ensure >10× colonies per potential sequence present within each library. Each library was isolated using an Endo-free Maxiprep kit (Qiagen). Library diversity was assessed by sequencing on an Illumina MiSeq 2×250 bp lane such that the entire mutated section was sequenced in both directions and a portion of the nonmutagenized section was sequenced as an internal control for background mutations.

### Lentiviral Preparation and Transduction.

SGCB cDNA subsection libraries (A-F) were packaged into lentivirus at the Hope Center Viral Core facility at Washington University by cotransfecting HEK293T cells (ATCC) with the transfer plasmid pool plus envelope and packaging vectors (pMD2.G, Addgene 12259 and psPAX2, 12260). For each pool, 4×106 cells were plated onto each of six 100 mm dishes and transfected with ug total plasmid (2.0:1.0:1.3 transfer/envelope/packaging ratio), using Lipofectamine 3000 (Thermo Fisher Scientific). Media were replaced after 6 hours, and viral supernatants were collected at 24 hours, filtered using a 0.45 mm filter (Millipore), and stored at −80° C. Viral titer was determined by qPCR and empirically estimated with a dilution series of transduced cells followed by FACS to measure the proportion of YFP-positive cells. For each biological replicate for each section, ADG-HEK cells were transduced with mutant library at low multiplicity of infection (<0.1), by applying 1.5 mL viral supernatant (with 8 mg/ml polybrene) to a 100 mm dish (TPP) containing approximately 5×106 cells, with three 100 mm dishes per replicate to maintain library diversity. After 72 hours, transduced cells were bulk sorted for the presence of YFP using a FACS Aria II (BD) into DMEM containing 100 U/mL penicillin and 100 g/mL streptomycin. Cells were grown for 5-7 days and then stained for cell surface expression of YFP-SGCB-HA or SGCA.

### Functional Selection.

Three measurements of function were obtained for each mutagenized section of SGCB. First, transduced cells were harvested during passaging to obtain a measurement of the frequency of each mutation present in the unselected pool of transduced cells. A sample of transduced cells after selection for YFP expression was obtained to measure the abundance of each mutation in YFP-positive cells compared with all transduced cells to measure the effect, if any, on total protein level. Third, YFP-positive sorted cells were further labeled with Alexa Fluor 647-conjugated antibody for FACS. Briefly, live cells were collected using trypsin-EDTA 0.05%. Thereafter, cells were blocked in blocking buffer (0.5% BSA-PBS) for 20 minutes on ice; half the cells were incubated with Alexa Fluor 647-conjugated anti-HA, and the other half were stained with Alexa Fluor 647-conjugated anti-α-sarcoglycan antibody on ice for 30 minutes. After incubation, cells were washed twice with PBS. The cells were fixed with 2% paraformaldehyde on ice for 20 minutes. Next, cells were resuspended in ClearFlow Sheath Fluid (Leinco Technologies) and sorted using FACS Aria II into bins according to the intensity of Alexa Fluor 647 signal. After the single cells were selected using forward and side scatter, a histogram of the FITC was created to select for YFP-positive cells. For those YFP-positive cells, a histogram of the Alexa Fluor 647 was further created and gates were drawn across the library based on red intensity: low (bin 1, 5%), medium low (bin 2, 5%), medium high (bin 3, 5%), and high (bin 4, 5%). Cells harboring single or double variants in β-sarcoglycan were labeled with Alexa Fluor 647-conjugated antibody in the same ways as described above. The single cells were gated and analyzed for YFP expression. Furthermore, the YFP-positive population was analyzed for the expression of Alexa Fluor 647 by MACS Flow Cytometer (Miltenyi Biotec). The data analysis was performed using FlowJo software (TreeStar Inc). Each assay was performed in duplicate starting from transduction.

### Amplification and Sequencing of Integrated SGCB.

After selections, genomic DNA was isolated from more than 500,000 cells using a DNeasy Blood and Tissue DNA extraction kit (Qiagen) and sequenced for the mutated region. For each sample, 16 replicate PCR reactions were performed and pooled, each with 200 ng genomic DNA as template. Mutated sections from integrated inserts were PCR amplified with NEBnext High-Fidelity polymerase (NEB). Each set of 16 replicate amplicons was pooled and purified with SPRI beads. Mutated sections were then amplified for 15 cycles using primers with 5′ overhangs of Illumina adapter handles and then secondarily amplified for cycles using dual-indexed illumina adapters. The resulting Illumina libraries contained inserts ranging from 200 to 240 bp, enabling full read coverage in both directions for 2×250 bp sequencing and more than 50 bp overlap for 2×150 bp sequencing. Illumina libraries were either sequenced using 2×250 bp flow cells on a MiSeq or 2×150 bp flow cells on a NovaSeq.

Variant enrichment, function scores, and pathogenicity predictions. Within each sample, raw read counts for each mutant codon were counted and per-mutation frequencies were calculated; any variant read counts under 50 per 1 million reads across the 4 sorted bins were removed from further analysis. For both HA-SGCB and SGCA, functional scores were taken as the log 10 ratio of the frequency in bin 4 (high expression) over that in bin 1 (low expression) and then normalized across chunks such that the functional scores for synonymous mutations in each chunk have an average of 0 and a standard deviation of 0.5. For amino acid substitutions represented by more than one equivalent codon substitution, the median was taken across those codons' functional scores to yield an amino acid-level score. The median score was taken for each amino acid functional score across biological replicates.

### Clinical and Population Variants.

SGCB missense variant classifications were obtained from ClinVar on Sep. 6, 2022; the Leiden database; or extracted from the literature. If a variant was called pathogenic in at least one source and never called benign or likely benign, it was herein considered pathogenic. Likely pathogenic variants were those only called likely pathogenic. Variants were called “conflicting” if the variant was reported both as benign or likely benign and also a VUS or pathogenic. Benign variants were defined as those list-ed as benign in at least one source or observed in the homozygous state in more than 1 individual in a population database. Population variation was obtained from the gnomAD database, combining whole-genome and -exome calls from versions 2.1.1 and 3.0. Population variant was also obtained from the UKBB exome sequenced data set (n=500,000).

### Bioinformatic Prediction Scores.

All SGCB missense variants were scored by REVEL, CADD, and PolyPhen-2. Scores were available only for missense variants reachable by single-base variants (SNVs); for amino acid substitutions that could arise from more than 1 SNV, the mean of those SNVs' scores was taken. These scores were only used for comparison to results from in vitro functional experiments and not used in the derivation of functional scores. Functional scores are entirely independent of these in silico predictions.

### SGC Gene Structure and Conservation.

The SGCB predicted structure was generated using AlphaFold2 using the multimer algorithm, including the SGCB, SGCA, SGCD, and SGCG protein sequences. The predicted protein structure of SGCB in the context of the other 3 SGC proteins was rendered with ChimeraX.

### Statistics.

Functional scores were defined as the log ratio of mutant read counts in cells with high cell surface SGC protein level and cells with low cell surface SGC protein level. For all quantitative measurements, mean±SEM are reported. Normal distribution of the sample sets was determined before application of 2-tailed Student's t test. A 2-sample Student's t test was used to compare the differences between 2 groups. Kaplan-Meier method was used to estimate the probability of survival, and the log-rank test was used to compare the overall survival difference between groups. Correlation between variables and significance was determined using linear regression with the Im function in R. All statistical tests were 2 sided, and the differences were considered significant when P was less than 0.05.

TABLE 1
Functional Score Pathogenicity Prediction

| | | | | | | | Functional | Functional | Consensus |
|---|---|---|---|---|---|---|---|---|---|
| AA1 | Position | AA2 | Variant | Codon | Consequence | Chunk | Score Prediction | Score | Pathogenicity |
| E | 28 | G | E28G | GAG | Missense | A | Functional | −0.33 | Benign |
| R | 30 | S | R30S | AGG | Missense | A | Functional | 0.04 | Benign |
| T | 126 | A | T126A | ACA | Missense | C | Functional | 0.13 | Benign |
| R | 267 | C | R267C | CGC | Missense | E | Functional | 0.16 | Benign |
| S | 31 | I | S31I | AGT | Missense | A | Functional | 0.39 | Benign |
| R | 223 | C | R223C | CGT | Missense | E | Functional | 0.4 | Benign |
| G | 315 | R | G315R | GGA | Missense | F | Functional | 0.03 | Conflicting |
| T | 265 | I | T265I | ACC | Missense | E | Functional | 0.58 | Conflicting |
| S | 210 | R | S210R | AGC | Missense | D | Non-Functional | −1.31 | Likely |
| | | | | | | | | | Pathogenic |
| M | 1 | L | M1L | ATG | Missense | A | Non-Functional | −1.26 | Likely |
| | | | | | | | | | pathogenic |
| I | 119 | N | I119N | ATC | Missense | C | Non-Functional | −1.15 | Likely |
| | | | | | | | | | Pathogenic |
| M | 247 | R | M247R | ATG | Missense | E | Non-Functional | −0.83 | Likely |
| | | | | | | | | | Pathogenic |
| I | 92 | S | I92S | ATT | Missense | B | Non-Functional | −2.23 | Pathogenic |
| M | 100 | K | M100K | ATG | Missense | B | Non-Functional | −2.02 | Pathogenic |
| M | 100 | L | M100K | ATG | Missense | B | Non-Functional | −2.02 | Pathogenic |
| R | 91 | L | R91L | CGC | Missense | B | Non-Functional | −1.93 | Pathogenic |
| R | 91 | P | R91P | CGC | Missense | B | Non-Functional | −1.76 | Pathogenic |
| G | 167 | S | G167S | GGC | Missense | D | Non-Functional | −1.64 | Pathogenic |
| I | 119 | F | I119F | ATC | Missense | C | Non-Functional | −1.3 | Pathogenic |
| T | 182 | P | T182P | ACA | Missense | D | Non-Functional | −1.25 | Pathogenic |
| T | 182 | A | T182A | ACA | Missense | D | Non-Functional | −1.25 | Pathogenic |
| L | 108 | R | L108R | CTT | Missense | B | Non-Functional | −1.09 | Pathogenic |
| M | 1 | T | M1T | ATG | Missense | A | Non-Functional | −1.08 | Pathogenic |
| T | 151 | R | T151R | ACA | Missense | C | Non-Functional | −1.06 | Pathogenic |
| M | 1 | I | M1I | ATG | Missense | A | Non-Functional | −1.06 | Pathogenic |
| I | 92 | T | I92T | ATT | Missense | B | Non-Functional | −1.03 | Pathogenic |
| M | 116 | V | M116V | ATG | Missense | C | Non-Functional | −1.02 | Pathogenic |
| G | 93 | A | G93A | GGA | Missense | B | Non-Functional | −0.97 | Pathogenic |
| V | 89 | M | V89M | GTG | Missense | B | Non-Functional | −0.96 | Pathogenic |
| S | 114 | F | S114F | TCT | Missense | C | Non-Functional | −0.89 | Pathogenic |
| L | 135 | W | L135W | TTG | Missense | C | Non-Functional | −0.88 | Pathogenic |
| G | 231 | S | G231S | GGT | Missense | E | Non-Functional | −0.88 | Pathogenic |
| S | 181 | R | S181R | AGC | Missense | D | Non-Functional | −0.87 | Pathogenic |
| G | 139 | D | G139D | GGC | Missense | C | Non-Functional | −0.84 | Pathogenic |
| G | 228 | E | G228E | GGA | Missense | E | Non-Functional | −0.84 | Pathogenic |
| S | 204 | P | S204P | TCT | Missense | D | Non-Functional | −0.83 | Pathogenic |
| Y | 184 | C | Y184C | TAT | Missense | D | Non-Functional | −0.8 | Pathogenic |
| S | 114 | P | S114P | TCT | Missense | C | Non-Functional | −0.77 | Pathogenic |
| R | 91 | C | R91C | CGC | Missense | B | Non-Functional | −0.72 | Pathogenic |
| F | 180 | L | F180L | TTC | Missense | D | Non-Functional | −0.42 | Conflicting |
| G | 96 | R | G96R | GGC | Missense | B | Non-Functional | −1.51 | VUS |
| S | 164 | I | S164I | AGT | Missense | D | Non-Functional | −1.26 | VUS |
| R | 227 | P | R227P | CGT | Missense | E | Non-Functional | −1.23 | VUS |
| G | 93 | E | G93E | GGA | Missense | B | Non-Functional | −1.2 | VUS |
| L | 153 | P | L153P | CTC | Missense | C | Non-Functional | −1.07 | VUS |
| C | 314 | R | C314R | TGT | Missense | F | Non-Functional | −1.01 | VUS |
| I | 144 | M | I144M | ATT | Missense | C | Non-Functional | −0.89 | VUS |
| K | 286 | E | K286E | AAG | Missense | F | Non-Functional | −0.77 | VUS |
| Q | 112 | L | Q112L | CAA | Missense | C | Non-Functional | −0.73 | VUS |
| C | 288 | F | C288F | TGC | Missense | F | Non-Functional | −0.71 | VUS |
| K | 159 | E | K159E | AAA | Missense | C | Non-Functional | −0.7 | VUS |
| H | 242 | P | H242P | CAC | Missense | E | Non-Functional | −0.67 | VUS |
| G | 274 | E | G274E | GGA | Missense | F | Non-Functional | −0.62 | VUS |
| R | 131 | Q | R131Q | CGA | Missense | C | Non-Functional | −0.6 | VUS |
| V | 127 | A | V127A | GTA | Missense | C | Non-Functional | −0.57 | VUS |
| D | 215 | V | D215V | GAT | Missense | D | Non-Functional | −0.56 | VUS |
| L | 198 | V | L198V | TTG | Missense | D | Non-Functional | −0.55 | VUS |
| D | 115 | H | D115H | GAC | Missense | C | Non-Functional | −0.53 | VUS |
| M | 247 | T | M247T | ATG | Missense | E | Non-Functional | −0.51 | VUS |
| K | 124 | E | K124E | AAA | Missense | C | Functional | −0.5 | VUS |
| T | 205 | P | T205P | ACT | Missense | D | Functional | −0.48 | VUS |
| N | 304 | S | N304S | AAC | Missense | F | Functional | −0.45 | VUS |
| G | 236 | D | G236D | GGC | Missense | E | Functional | −0.43 | VUS |
| I | 178 | T | I178T | ATC | Missense | D | Functional | −0.41 | VUS |
| A | 76 | D | A76D | GCT | Missense | B | Functional | −0.4 | VUS |
| N | 140 | S | N140S | AAC | Missense | C | Functional | −0.38 | VUS |
| C | 307 | R | C307R | TGC | Missense | F | Functional | −0.37 | VUS |
| W | 282 | C | W282C | TGG | Missense | F | Functional | −0.37 | VUS |
| A | 7 | E | A7E | GCG | Missense | A | Functional | −0.36 | VUS |
| P | 121 | T | P121T | CCT | Missense | C | Functional | −0.36 | VUS |
| N | 132 | K | N132K | AAT | Missense | C | Functional | −0.36 | VUS |
| T | 209 | I | T209I | ACC | Missense | D | Functional | −0.34 | VUS |
| R | 60 | G | R60G | AGA | Missense | B | Functional | −0.33 | VUS |
| T | 163 | A | T163A | ACA | Missense | D | Functional | −0.32 | VUS |
| Q | 276 | L | Q276L | CAG | Missense | F | Functional | −0.3 | VUS |
| F | 170 | C | F170C | TTT | Missense | D | Functional | −0.28 | VUS |
| T | 301 | A | T301A | ACC | Missense | F | Functional | −0.27 | VUS |
| N | 158 | K | N158K | AAC | Missense | C | Functional | −0.26 | VUS |
| N | 63 | S | N63S | AAT | Missense | B | Functional | −0.25 | VUS |
| R | 51 | C | R51C | CGT | Missense | A | Functional | −0.24 | VUS |
| M | 168 | I | M168I | ATG | Missense | D | Functional | −0.24 | VUS |
| S | 154 | G | S154G | AGT | Missense | C | Functional | −0.24 | VUS |
| Q | 201 | P | Q201P | CAA | Missense | D | Functional | −0.23 | VUS |
| M | 305 | V | M305V | ATG | Missense | F | Functional | −0.22 | VUS |
| K | 152 | R | K152R | AAG | Missense | C | Functional | −0.22 | VUS |
| N | 37 | S | N37S | AAC | Missense | A | Functional | −0.21 | VUS |
| S | 125 | N | S125N | AGC | Missense | C | Functional | −0.2 | VUS |
| P | 46 | L | P46L | CCG | Missense | A | Functional | −0.2 | VUS |
| T | 205 | S | T205S | ACT | Missense | D | Functional | −0.16 | VUS |
| T | 175 | N | T175N | ACT | Missense | D | Functional | −0.16 | VUS |
| E | 133 | G | E133G | GAA | Missense | C | Functional | −0.14 | VUS |
| S | 273 | R | S273R | AGT | Missense | F | Functional | −0.14 | VUS |
| S | 164 | G | S164G | AGT | Missense | D | Functional | −0.13 | VUS |
| M | 168 | V | M168V | ATG | Missense | D | Functional | −0.11 | VUS |
| K | 61 | E | K61E | AAG | Missense | B | Functional | −0.08 | VUS |
| S | 270 | N | S270N | AGT | Missense | E | Functional | −0.08 | VUS |
| S | 279 | G | S279G | AGT | Missense | F | Functional | −0.07 | VUS |
| L | 191 | W | L191W | TTG | Missense | D | Functional | −0.06 | VUS |
| R | 267 | L | R267L | CGC | Missense | E | Functional | −0.05 | VUS |
| N | 316 | S | N316S | AAC | Missense | F | Functional | −0.05 | VUS |
| K | 286 | N | K286N | AAG | Missense | F | Functional | −0.03 | VUS |
| A | 5 | E | A5E | GCG | Missense | A | Functional | −0.03 | VUS |
| I | 81 | V | I81V | ATA | Missense | B | Functional | −0.03 | VUS |
| N | 15 | S | N15S | AAT | Missense | A | Functional | −0.02 | VUS |
| N | 211 | S | N211S | AAT | Missense | D | Functional | −0.02 | VUS |
| S | 38 | R | S38R | AGT | Missense | A | Functional | −0.01 | VUS |
| V | 145 | A | V145A | GTT | Missense | C | Functional | −0.01 | VUS |
| R | 51 | H | R51H | CGT | Missense | A | Functional | 0 | VUS |
| K | 20 | T | K20T | AAG | Missense | A | Functional | 0 | VUS |
| D | 311 | N | D311N | GAC | Missense | F | Functional | 0.01 | VUS |
| A | 5 | V | A5V | GCG | Missense | A | Functional | 0.01 | VUS |
| M | 22 | V | M22V | ATG | Missense | A | Functional | 0.01 | VUS |
| L | 84 | P | L84P | CTT | Missense | B | Functional | 0.02 | VUS |
| G | 148 | R | Q148R | CAA | Missense | C | Functional | 0.02 | VUS |
| H | 120 | Y | H120Y | CAC | Missense | C | Functional | 0.03 | VUS |
| R | 284 | C | R284C | CGC | Missense | F | Functional | 0.03 | VUS |
| I | 82 | V | I82V | ATA | Missense | B | Functional | 0.04 | VUS |
| I | 255 | V | I255V | ATC | Missense | E | Functional | 0.07 | VUS |
| S | 310 | L | S310L | TCA | Missense | F | Functional | 0.07 | VUS |
| C | 67 | R | C67R | TGT | Missense | B | Functional | 0.08 | VUS |
| A | 42 | T | A42T | GCT | Missense | A | Functional | 0.1 | VUS |
| G | 280 | S | G280S | GGT | Missense | F | Functional | 0.11 | VUS |
| T | 138 | I | T138I | ACT | Missense | C | Functional | 0.11 | VUS |
| I | 239 | V | 1239V | ATT | Missense | E | Functional | 0.12 | VUS |
| I | 166 | V | 1166V | ATC | Missense | D | Functional | 0.13 | VUS |
| S | 125 | C | S125C | AGC | Missense | C | Functional | 0.13 | VUS |
| G | 56 | R | G56R | GGG | Missense | B | Functional | 0.14 | VUS |
| G | 11 | R | Q11R | CAG | Missense | A | Functional | 0.14 | VUS |
| I | 239 | T | I239T | ATT | Missense | E | Functional | 0.15 | VUS |
| A | 9 | V | A9V | GCA | Missense | A | Functional | 0.15 | VUS |
| R | 284 | H | R284H | CGC | Missense | F | Functional | 0.16 | VUS |
| Q | 11 | E | Q11E | CAG | Missense | A | Functional | 0.17 | VUS |
| M | 243 | I | M243I | ATG | Missense | E | Functional | 0.18 | VUS |
| M | 262 | T | M262T | ATG | Missense | E | Functional | 0.19 | VUS |
| Q | 11 | H | Q11H | CAG | Missense | A | Functional | 0.19 | VUS |
| M | 305 | I | M305I | ATG | Missense | F | Functional | 0.19 | VUS |
| N | 246 | Y | N246Y | AAT | Missense | E | Functional | 0.19 | VUS |
| T | 294 | M | T294M | ACG | Missense | F | Functional | 0.19 | VUS |
| A | 3 | E | A3E | GCA | Missense | A | Functional | 0.2 | VUS |
| R | 223 | H | R223H | CGT | Missense | E | Functional | 0.2 | VUS |
| R | 23 | H | R23H | CGT | Missense | A | Functional | 0.2 | VUS |
| V | 298 | M | V298M | GTG | Missense | F | Functional | 0.21 | VUS |
| A | 6 | V | A6V | GCG | Missense | A | Functional | 0.22 | VUS |
| T | 126 | I | T126I | ACA | Missense | C | Functional | 0.24 | VUS |
| D | 281 | N | D281N | GAC | Missense | F | Functional | 0.25 | VUS |
| T | 266 | I | T266I | ACC | Missense | E | Functional | 0.26 | VUS |
| A | 7 | T | A7T | GCG | Missense | A | Functional | 0.27 | VUS |
| E | 10 | G | E10G | GAA | Missense | A | Functional | 0.28 | VUS |
| I | 256 | M | I256M | ATC | Missense | E | Functional | 0.29 | VUS |
| G | 278 | D | G278D | GGT | Missense | F | Functional | 0.31 | VUS |
| L | 64 | S | L64S | TTA | Missense | B | Functional | 0.31 | VUS |
| H | 190 | R | H190R | CAT | Missense | D | Functional | 0.34 | VUS |
| N | 33 | D | N33D | AAT | Missense | A | Functional | 0.4 | VUS |
| A | 4 | P | A4P | GCG | Missense | A | Functional | 0.44 | VUS |
| A | 251 | V | A251V | GCG | Missense | E | Functional | 0.46 | VUS |
| E | 240 | K | E240K | GAA | Missense | E | Functional | 0.46 | VUS |
| V | 232 | I | V232I | GTA | Missense | E | Functional | 0.47 | VUS |
| R | 23 | C | R23C | CGT | Missense | A | Functional | 0.49 | VUS |
| M | 243 | V | M243V | ATG | Missense | E | Functional | 0.49 | VUS |
| T | 186 | A | T186A | ACT | Missense | D | Functional | 0.54 | VUS |
| R | 227 | H | R227H | CGT | Missense | E | Functional | 0.55 | VUS |
| I | 47 | T | I47T | ATT | Missense | A | Functional | 0.58 | VUS |
| N | 253 | S | N253S | AAC | Missense | E | Functional | 0.59 | VUS |
| K | 41 | E | K41E | AAA | Missense | A | Functional | 0.61 | VUS |
| F | 233 | S | F233S | TTC | Missense | E | Functional | 0.62 | VUS |
| Y | 123 | S | Y123S | TAT | Missense | C | Functional | 0.68 | VUS |
| Y | 44 | C | Y44C | TAC | Missense | A | Functional | 0.8 | VUS |
| G | 244 | D | G244D | GGT | Missense | E | Functional | 0.99 | VUS |

What is claimed is:

1. A method of generating a limb-girdle muscular dystrophy (LGMD) functional score, the method comprising:
providing a cell sample from the subject;
performing a single amino acid mutagenesis on a target protein in the cell sample;
performing a deep mutation scan (DMS) on the target protein following the single amino acid mutagenesis;
determining a mutant reads high expression, defined as a number of mutant read counts in cells of the cell sample having a high cell surface level of the target protein;
determining a mutant reads low expression, defined as a number of mutant read counts in cells of the cell sample having a low cell surface level of the target protein; and
generating a LGMD functional score for the target protein based on the mutant reads high expression and the mutant reads low expression.

2. The method of claim 1, wherein the target protein is a SGC protein.

3. The method of claim 2, wherein the SGC protein is a SGCB protein.

4. The method of claim 1, wherein the single amino acid mutagenesis is derived from an amino acid change requiring at least one nucleotide change.

5. The method of claim 1, wherein the LGMD functional score is generated by:
$$Functional score = {log}_{10} \frac{mutant reads high expression}{mutant reads low expression} .$$

6. The method of claim 5, wherein the LGMD functional score ranges from a score of −3 to a score of 1.5.

7. The method of claim 6, wherein a negative LGMD functional score indicates a deleterious variant and a positive LGMD functional score indicates a neutral variant.

8. The method of claim 7, wherein the LGMD functional score of less than −2 indicates severe LGMD and the LGMD functional score of greater than −2 indicates mild LGMD.

9. A method of treating limb-girdle muscular dystrophy (LGMD) in a subject in need thereof, the method comprising:
providing a cell sample from the subject;
performing a single amino acid mutagenesis on a target protein in the cell sample;
performing a deep mutation scan (DMS) on the target protein following the single amino acid mutagenesis;
determining a mutant reads high expression, defined as a number of mutant read counts in cells of the cell sample having a high cell surface level of the target protein;
determining a mutant reads low expression, defined as a number of mutant read counts in cells of the cell sample having a low cell surface level of the target protein;
generating a LGMD functional score for the target protein based on the mutant reads high expression and the mutant reads low expression; and
determining a gene therapy treatment for the subject based on the LGMD functional score.

10. The method of claim 9, wherein the target protein is a SGC protein.

11. The method of claim 10, wherein the SGC protein is a SGCB protein.

12. The method of claim 9, wherein the single amino acid mutagenesis is derived from an amino acid change requiring at least one nucleotide change.

13. The method of claim 9, wherein the LGMD functional score is generated by:
$$Functional score = {log}_{10} \frac{mutant reads high expression}{mutant reads low expression} .$$

14. The method of claim 13, wherein the LGMD functional score ranges from a score of −3 to a score of 1.5.

15. The method of claim 14, wherein a negative LGMD functional score indicates a deleterious variant and a positive LGMD functional score indicates a neutral variant.

16. The method of claim 15, wherein the LGMD functional score of less than −2 indicates severe LGMD and the LGMD functional score of greater than −2 indicates mild LGMD.

17. A method for determining limb-girdle muscular dystrophy (LGMD) severity in a subject in need thereof, the method comprising:
providing a cell sample from the subject;
performing a single amino acid mutagenesis on a target protein in the cell sample;
performing a deep mutation scan (DMS) on the target protein following the single amino acid mutagenesis;
determining a mutant reads high expression, defined as a number of mutant read counts in cells of the cell sample having a high cell surface level of the target protein;
determining a mutant reads low expression, defined as a number of mutant read counts in cells of the cell sample having a low cell surface level of the target protein;
generating a LGMD functional score for the target protein based on the mutant reads high expression and the mutant reads low expression; and
determining a LGMD severity for the subject based on the LGMD functional score.

18. The method of claim 17, wherein the LGMD severity comprises at least one of an age at loss of ambulation LGMD severity and an age at onset LGMD severity.

19. The method of claim 17, wherein the LGMD functional score is generated by:
$$Functional score = {log}_{10} \frac{mutant reads high expression}{mutant reads low expression} .$$

20. The method of claim 19, wherein:
the LGMD functional score ranges from a score of −3 to a score of 1.5;
a negative LGMD functional score indicates a deleterious variant and a positive LGMD functional score indicates a neutral variant; and
the LGMD functional score of less than −2 indicates severe LGMD and the LGMD functional score of greater than −2 indicates mild LGMD.
